(19) United States
(12) Patent Application Publication
(10) Pub. No.: US 2022/0214359 A1
(43) Pub. Date: Jul. 7, 2022

(54) Elucidating a Proteomic Signature for the Detection of Intracerebral Aneurysms

(71) Applicant: Icahn School of Medicine at Mount Sinai, New York, NY (US)

(72) Inventors: Dominic A. Nistal, New York, NY (US); J Mocco, New York, NY (US); Christopher P. Killner, New York, NY (US)

(21) Appl. No.: 17/606,981

(22) PCT Filed: May 1, 2020
(86) PCT No.: PCT/US20/31159

§ 371 Date: Oct. 27, 2021

Related U.S. Application Data
(60) Provisional application No. 62/841,725, filed on May 1, 2019.

(51) Int. Cl. G01N 33/68; A61B 5/02

(52) U.S. Cl. CPC G01N 33/6896; G01N 2800/60; G01N 2800/329; A61B 5/02014

(57) ABSTRACT

Systems and methods for detecting an intracranial aneurysm in a test subject are provided. Liquid biological samples are obtained from the test subject, each liquid biological sample comprising a plurality of protein analytes. Liquid biological samples are analyzed using an immunoassay, obtaining a test dataset comprising a plurality of abundance measures. Each abundance measure corresponds to a respective protein analyte in the plurality of protein analytes. The test dataset is inputted into a trained classifier, obtaining an indication from the trained classifier that the subject has an intracranial aneurysm, based at least in part on the plurality of abundance measures for the test subject in the test dataset.

### CROSS REFERENCE TO RELATED APPLICATION

This application claims priority to U.S. Provisional Patent Application No. 62/841,725, entitled “Elucidating a Proteomic Signature for The Detection of Intracerebral Aneurysms,” file May 1, 2020, which is hereby incorporated by reference.

### TECHNICAL FIELD

The present disclosure generally relates to detection of intracranial aneurysms using protein analytes.

### BACKGROUND

Intracranial aneurysms (IAs) are cerebrovascular lesions characterized by a weakening of the intravascular wall. Aneurysm pathogenesis, which appears to be an inflammatory process, results in an abnormal vessel dilatation and risk of rupture. See, Chen et al., Neurol India 61, 293-299 (2013); van der Voet et al., Am J Hum Genet 74, 564-571 (2004); and Witkowska et al., Arch Immunol Ther Exp (Warsz) 57, 137-140 (2009). Subarachnoid hemorrhage (SAH), one of the most feared consequences of IAs, occurs when a saccular aneurysm ruptures. SAH is often fatal, with a mortality rate estimated around 25%-50%. See, Barcelos et al., Neurocrit Care 18, 234-244 (2013); Fontanella et al., Neurosurgery 60, 668-672 (2007); Liu et al., Journal of International Medical Research 41, 1079-1087 (2013); McColgan et al., Journal of Neurosurgery 112, 714-721 (2010); Pannu et al., Journal of Neurosurgery 103, 92-96 (2005); Pera et al., Stroke 41, 224-231 (2010), and Sandalcioglu et al., Neurosurgical Review 29, 26-29 (2006).

Though IAs are reported to occur in 0.4%-6.0% of the population, diagnosis usually occurs when the patient becomes symptomatic. Of note, several studies have shown that diagnosis and surgical treatment of an IA prior to rupture dramatically reduces the rates of mortality to 0.0%-2.5%. See, Baker et al., Neurosurgery 37, 56-62 (1995); Kassam et al., Neurosurgery 54, 1199-1212 (2004); and Phillips et al., Neurosurgery 40, 1112-1117 (1997). Unfortunately, studies have suggested that approximately 90% of unruptured aneurysms are asymptomatic, leading to delayed diagnosis and treatment. See, Keedy, McGill Journal of Medicine 9, 141-146 (2006). As such, there is an important unmet medical need to improve our ability to detect the presence of an aneurysm as early as possible.

Imaging is currently the gold standard for diagnosis of cerebrovascular pathophysiology. See, Hussain et al., World Neurosurg 84, 1473-1483 (2015). The top detection methods include intra-arterial digital subtraction angiography, computed tomography angiography, and magnetic resonance angiography. However, these characterizations are typically only available in specialized centers and are associated with high costs. See, Jethwa et al., Neurosurgery 72, 511-519; discussion 519 (2013). Furthermore, angiograms are invasive and have adverse risks such as subarachnoid hemorrhage, incision infection, and allergic reaction. See, Cloft et al., Stroke 30, 317-320 (1999). Therefore, the current standard of aneurysm detection is not suitable for population-based screening and will likely never become part of routine clinical assessment. Thus, a blood-based measure that accurately reflects saccular aneurysm pathology, ideally at the preclinical phase, would be a significant advantage to aneurysm treatment and subarachnoid hemorrhage prevention.

Given the above background, what is needed in the art are improved systems and methods for non-invasive and accurate early detection of intracranial aneurysms.

### SUMMARY

Accordingly, there is a demand for accurate and non-invasive methods and systems for screening and early detection of intracranial aneurysms, especially asymptomatic and/or unruptured aneurysms. The present disclosure addresses these needs, for example, by providing herein a method for detecting an intracranial aneurysm in a test subject, and a classification method for training a classifier to provide an indication that a subject has an intracranial aneurysm.

One aspect of the present disclosure provides a method for detecting an intracranial aneurysm in a test subject. The method comprises obtaining one or more liquid biological samples from the test subject, where each liquid biological sample in the one or more liquid biological samples comprises a plurality of protein analytes. The method further comprises analyzing each liquid biological sample in the one or more liquid biological samples using an immunoassay, thus obtaining a test dataset comprising a plurality of abundance measures, where each abundance measure in the plurality of abundance measures corresponds to a respective protein analyte in the plurality of protein analytes in each respective liquid biological sample in the one or more liquid biological samples. The method further comprises inputting the test dataset into a trained classifier, thus obtaining an indication from the trained classifier that the subject has an intracranial aneurysm, based at least in part on the plurality of abundance measures for the test subject in the test dataset.

In some embodiments, the analyzing each liquid biological sample using an immunoassay comprises measuring the abundance of one or more protein analytes selected from a predefined panel of protein analytes. In some embodiments, the predefined panel of protein analytes comprises one or more analytes selected from Table 1. In some embodiments, the predefined panel of protein analytes comprises one or more analytes selected from Table 2.

In some embodiments, the immunoassay is a high-throughput multiplex proximity extension immunoassay.

In some embodiments, the test dataset further comprises a first label indicating a corresponding first covariate for the test subject, the indication from the trained classifier that the subject has an intracranial aneurysm is further based on the first covariate, and the corresponding first covariate is selected from the group consisting of an age of the test subject; a sex of the test subject; a hypertension status; a hyperlipidemia status; a presence or absence of diabetes mellitus type II; and a smoking history.

In some embodiments, the test dataset is pre-processed by normalization of the plurality of abundance measures prior to the inputting the test dataset into the trained classifier.

In some embodiments, the test dataset is processed, prior to the inputting the test dataset into the trained classifier, by removing from the dataset one or more protein analytes that fail to meet one or more selection criteria. In some embodiments, the one or more selection criteria is a threshold limit of detection. In some embodiments, the one or more selection criteria is inclusion in a predefined panel of protein analytes.

In some embodiments, the indication comprises a probability that the subject has an intracranial aneurysm and a prediction of a size of an intracranial aneurysm.

In some embodiments, the trained classifier is a neural network algorithm, a support vector machine algorithm, a Naïve Bayes algorithm, a decision tree algorithm, an unsupervised clustering model algorithm, a supervised clustering model algorithm, or a regression model.

In some embodiments, the test subject is a human. In some embodiments, the test subject has an unruptured intracranial aneurysm. In some embodiments, each liquid biological sample in the one or more liquid biological samples is a blood sample. In some embodiments, each abundance measure in the plurality of abundance measures is a relative protein concentration.

In some embodiments, the obtaining one or more liquid biological samples from the test subject is performed by venipuncture.

In some embodiments, the method further comprises applying a treatment regimen to the test subject based at least in part, on the indication. In some embodiments, the treatment regimen comprises applying an agent for intracranial aneurysm. In some embodiments, the agent for intracranial aneurysm is a hormone, an immune therapy, radiography, or a drug.

In some embodiments, the subject has been treated with an agent for intercranial aneurysm and the method further comprises using the indication to evaluate a response of the test subject to the agent for intercranial aneurysm. In some embodiments, the agent for intercranial aneurysm is a hormone, an immune therapy, radiography, or a drug.

In some embodiments, the subject has been treated with an agent for intercranial aneurysm and the method further comprises using the indication to determine whether to intensify or discontinue the agent for intercranial aneurysm in the test subject.

In some embodiments, the subject has been subjected to a surgical intervention to address the intercranial aneurysm and the method further comprises using the indication to assess a success of the surgical intervention.

Another aspect of the present disclosure provides a classification method, at a computer system having one or more processors, and memory storing one or more programs for execution by the one or more processors. The method comprises, for each training subject in a plurality of training subjects, where each training subject in the plurality of training subjects is distinguished as having a first diagnostic status corresponding to either a presence of an intracranial aneurysm or an absence of an intracranial aneurysm, obtaining one or more liquid biological samples from each respective training subject, thus obtaining a plurality of liquid biological samples, where each liquid biological sample comprises a plurality of protein analytes. The method further comprises analyzing each liquid biological sample in the plurality of liquid biological samples using an immunoassay, thus obtaining a first dataset. The first dataset comprises, for each training subject in the plurality of training subjects (i) a first label indicating the corresponding first diagnostic status of the respective subject and (ii) a plurality of abundance measures, where each abundance measure in the plurality of abundance measures corresponds to a respective protein analyte in the plurality of protein analytes in each respective liquid biological sample in the one or more liquid biological samples. The method further comprises training an untrained or partially untrained classifier with the first dataset, thus obtaining a trained classifier that provides an indication that a subject has an intracranial aneurysm, based at least in part on a plurality of abundance measures for a corresponding plurality of protein analytes in one or more liquid biological samples of the subject.

In some embodiments, the analyzing each liquid biological sample using an immunoassay comprises measuring the abundance of one or more protein analytes selected from a predefined panel of protein analytes. In some embodiments, the predefined panel of protein analytes comprises one or more analytes selected from Table 1. In some embodiments, the predefined panel of protein analytes comprises one or more analytes selected from Table 2.

In some embodiments, the immunoassay is a high-throughput multiplex proximity extension immunoassay.

In some embodiments, the plurality of training subjects comprises a first subset of training subjects and a second subset of training subjects; each respective training subject in the first subset of training subjects has a first diagnostic status corresponding to a presence of an intracranial aneurysm; each respective training subject in the second subset of training subjects has a first diagnostic status corresponding to an absence of an intracranial aneurysm; and the number of training subjects in the first subset of training subjects is equal to the number of training subjects in the second subset of training subjects.

In some embodiments, the first dataset is pre-processed by normalization of the plurality of abundance measures prior to the training the untrained or partially untrained classifier with the first dataset.

In some embodiments, the first dataset is processed, prior to the training the untrained or partially untrained classifier with the first dataset, by removing from the dataset one or more protein analytes that fail to meet one or more selection criteria. In some embodiments, the one or more selection criteria is a threshold limit of detection. In some embodiments, the one or more selection criteria is inclusion in a predefined panel of protein analytes.

In some embodiments, the one or more selection criteria is a threshold p-value, where the p-value for each one or more protein analyte is (i) determined using a significance test and (ii) calculated over the plurality of abundance measures corresponding to the respective protein analyte across the plurality of training subjects. In some embodiments, the significance test is a univariate linear regression model, a univariate logistic regression model, a multivariate linear regression model, a multivariate logistic regression model, a chi-squared test, Fishers Exact test, Student's t-test, or a binary proportional test. In some embodiments, the threshold p-value is 0.05. In some embodiments, the threshold p-value is 0.0001.

In some embodiments, the first dataset further comprises, for each subject in the plurality of subjects, a second label indicating a corresponding second diagnostic status, where the second diagnostic status is selected from the group consisting of a size of an intracranial aneurysm; a location of an intracranial aneurysm; a presence or absence of aneurysmal rupture; a saccular aneurysm; an endovascular treatment status for an intracranial aneurysm; an open treatment status for an intracranial aneurysm; an age of a training subject; a sex of a training subject; a hypertension status; a hyperlipidemia status; a presence or absence of diabetes mellitus type II; and a smoking history.

In some such embodiments, the indication from the trained classifier that a subject has an intracranial aneurysm is further based on the second diagnostic status. In some alternative embodiments, the trained classifier further provides an indication that a subject has the second diagnostic status. In some embodiments, the indication comprises a probability that a subject has an intracranial aneurysm and a prediction of a size of an intracranial aneurysm.

In some embodiments, the trained classifier is a neural network algorithm, a support vector machine algorithm, a Naïve Bayes algorithm, a decision tree algorithm, an unsupervised clustering model algorithm, a supervised clustering model algorithm, or a regression model.

In some embodiments, prior to the training the untrained or partially untrained classifier, the performance of the untrained or partially untrained classifier is validated on the first dataset using k-fold cross validation. In some embodiments, k is between 2 and 60.

In some embodiments, each training subject in the plurality of training subjects is a human. In some embodiments, each liquid biological sample in the plurality of liquid biological samples is a blood sample. In some embodiments, each abundance measure in the plurality of abundance measures is a relative protein concentration. In some embodiments, the obtaining one or more liquid biological samples from each respective training subject is performed by venipuncture.

Another aspect of the present disclosure further provides a device comprising one or more processors, and memory storing one or more programs for execution by the one or more processors, the one or more programs comprising instructions to perform any of the disclosed methods and embodiments.

Another aspect of the present disclosure further provides a non-transitory computer readable storage medium and one or more computer programs embedded therein, the one or more computer programs comprising instructions which, when executed by a computer system, cause the computer system to perform any of the disclosed methods and embodiments.

Any embodiment disclosed herein when applicable can be applied to any aspect.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 illustrates a block diagram of an example computing device, in accordance with some embodiments of the present disclosure.

FIGS. 2A-2B collectively provide a flow chart of processes and features for detecting an intracranial aneurysm in a test subject, in which optional blocks are indicated with dashed boxes, in accordance with some embodiments of the present disclosure.

FIGS. 3A-3B collectively provide a flow chart of processes and features for training a classifier to detect an intracranial aneurysm in a subject, in which optional blocks are indicated with dashed boxes, in accordance with some embodiments of the present disclosure.

FIG. 4 illustrates experimental Receiver Operating Characteristics (ROC) curves for evaluating accuracy of the disclosed method for the detection of intracranial aneurysms, in accordance with some embodiments of the present disclosure.

FIGS. 5A and 5B illustrate the relative abundance (upregulated 5A, downregulated 5B) of a plurality of protein analytes in liquid biological samples obtained from subjects with and without intracranial aneurysms, in accordance with some embodiments of the present disclosure.

FIG. 6 provides demographic and clinical information of intracranial aneurysm patient and control subject cohorts, in accordance with some embodiments of the present disclosure.

Like reference numerals refer to corresponding parts throughout the several views of the drawings. The drawings are not drawn to scale.

### DETAILED DESCRIPTION

### Benefit

Early detection of unruptured intracranial aneurysms (IAs) provides several advantages to clinical management, including the monitoring and treatment of unruptured aneurysms, thus reducing the incidence of aneurysm subarachnoid hemorrhage. For example, improved early detection of unruptured aneurysms could enhance the triage of patients presenting with symptoms concerning for aneurysm formation and growth, and could also reduce our reliance on neuroimaging for aneurysm monitoring.

One method for addressing this need is the identification of serum protein biomarkers that correlate with the presence and size of IAs. Previous studies in the field have largely focused on identifying individual biomarkers to facilitate prediction of outcomes following SAH or vasospasm. See, Shi et al., Stroke 40, 1252-1261 (2009); Jung et al., Stroke Res Treat 2013, 560305 (2013); Nakaoka et al., Stroke 45, 2239-2245 (2014); Przybycien-Szymanska and Ashley, J Stroke Cerebrovasc Dis 24, 1453-1464 (2015); Siman et al., PLoS One 6, e28938 (2011); Rodriguez-Rodriguez et al., J Neurol Sci 341, 119-127 (2014); Chou et al., Transl Stroke Res 2, 600-607 (2011); and Lad et al., J Stroke Cerebrovasc Dis 21, 30-41 (2012). Despite their perceived utility in predicting patient prognosis following aneurysmal rupture, however, the biomarkers identified in these studies do not help improve early detection or prevention of IA rupture. Additionally, while a diagnostic signature has been pursued by studies focusing on the peripheral vasculature, particularly with abdominal aortic aneurysms, no studies to date have sought to address this unmet need for IAs. See, Li et al., BMC Cardiovasc Disord 18, 60 (2018); Bylund and Henriksson, Am J Cardiovasc Dis 5, 140-145 (2015); Wallinder et al., Clin Transl Sci 5, 56-59 (2012); Gamberi et al., Mot Biosyst 7, 2855-2862 (2011); Pulinx et al., Eur J Vasc Endovasc Surg 42, 563-570 (2011); Acosta-Martin et al., PLoS One 6, e28698 (2011); Nordon et al., Nat Rev Cardiol 8, 92-102 (2011); and Spadaccio et al., Cardiovasc Pathol 21, 283-290 (2012). As such, predicting the presence, size, and stage of IAs is impeded due to limited understanding of the underlying pathophysiological processes that drive aneurysm formation and growth. There is a significant unmet clinical need for identifying a comprehensive signature in patient blood to accurately predict the presence of an unruptured IA and improve early detection and rupture prevention.

Traditionally, studies attempting to discover serum biomarkers for unruptured IAs have relied on a limited selection of diagnostic screening assays to quantify the levels of specific serum markers in experimental and control populations. While these candidate-based discovery methods have helped characterize individual biomarkers associated with aneurysm presence, they are slow, expensive, and lack the capacity for a more universal basis for discovering novel disease-associated serum biomarkers in biologically complex patient samples. See, Solier and Langen, Proteomics 14, 774-783 (2014).

Importantly, the technological landscape for biomarker discovery has been rapidly advancing over the past few years. High-throughput, precision profiling methods are promising solutions because they can provide a more objective view of the biochemical compositions across multiple clinical specimens and can reveal unexpected alterations in proteomic profiles. The intricate and often interacting effects of several molecular players in a given pathophysiological mechanism highlights the suitability of using a validated signature of biomarkers to accurately identify and classify cases of the present condition.

For example, in the realm of unruptured IAs, identifying a proteomic signature of serum protein biomarkers that correlate with the presence and size of IAs can improve staging and prognostication techniques to better inform appropriate management and treatment for patients with an unruptured aneurysm. In addition, the extensive proteomic coverage of critical neurological and inflammatory processes in this study may offer new insights into the pathogenesis of IAs and may suggest new candidate molecular targets for therapeutic intervention. Accurate prediction using such a proteomic signature for IA, combined with an understanding of the role of these biomarkers in IA pathophysiology, will be critical to enhance the ability of clinicians to treat and manage patients with unruptured IAs and reduce the morbidity and mortality associated with this serious cerebrovascular lesions.

Furthermore, the proteomic signature can be utilized in combination with patient outcomes to enhance prediction algorithms to more accurately determine which patients are at a greater risk of rupture and which patients will benefit most from various therapeutic modalities. Such serum biomarker signatures can be used in clinically relevant blood tests to facilitate early detection and mortality reduction. For instance, an actionable and affordable blood test for aneurysm discovery can provide for the detection of unruptured IAs using a blood-based measure, thus offering early, accessible diagnosis and future aneurysm management.

In view of the abovementioned benefits, the present disclosure provides a high-precision, proteomic-level method to identify and use a predictive biomarker signature for the screening and diagnosis of unruptured IAs using patient-derived serum samples. The disclosed methods comprise analysis of the peripheral blood proteome in patients with unruptured IAs to identify the relative abundance of protein biomarkers (e.g., upregulated or downregulated) compared to healthy controls, with a goal of identifying potential therapeutic agents to prevent aneurysm formation or progression.

More particularly, the present disclosure provides systems and methods for detecting an intracranial aneurysm in a test subject, such as a patient. The method comprises obtaining one or more liquid biological samples (e.g., serum samples) from the test subject, each liquid biological sample comprising a plurality of protein analytes. Liquid biological samples are analyzed using an immunoassay, such as a high-throughput multiplex proximity extension immunoassay, thus obtaining a test dataset comprising a plurality of abundance measures (e.g., relative protein concentrations). Each abundance measure corresponds to a respective protein analyte in the plurality of protein analytes in each respective liquid biological sample in the one or more liquid biological samples. The test dataset is then inputted into a trained classifier (e.g., a support vector machine or a multivariate logistic regression model), obtaining an indication from the trained classifier that the subject has an intracranial aneurysm (e.g., a presence or absence of an unruptured IA and/or a size of an unruptured IA), where the indication is based at least in part on the plurality of abundance measures for the test subject in the test dataset. Using the methods disclosed herein, the detection of an IA in the respective patient can then be used to select a treatment regimen, such as a therapeutic agent (e.g., a hormone, an immune therapy, radiography, or a drug), which is applied to the patient. In some implementations, the detection of an IA is used to evaluate a patient response (e.g., a presence or absence of an IA and/or a reduction in size of an IA) following a treatment and/or a surgical intervention. The evaluation of such response can then be used to select an appropriate action following the treatment and/or surgical intervention, such as an intensification or a discontinuation of the treatment.

The present disclosure further provides systems and methods for classification of an intracranial aneurysm. The method comprises obtaining one or more liquid biological samples (e.g., serum samples) from each respective training subject in a plurality of training subjects, thus obtaining a plurality of liquid biological samples. Each training subject in the plurality of training subjects is distinguished as having a first diagnostic status corresponding to either a presence of an intracranial aneurysm (e.g., a clinical subject or a patient) or an absence of an intracranial aneurysm (e.g., a control subject). Each liquid biological sample comprises a plurality of protein analytes. The liquid biological samples are analyzed using an immunoassay, thus obtaining a first dataset (e.g., a training dataset) comprising, for each training subject, a first label indicating whether the respective subject has a presence or absence of an intracranial aneurysm (e.g., whether the subject is an IA or a control subject). The training dataset further comprises a plurality of abundance measures (e.g., relative protein concentrations), where each abundance measure corresponds to a respective protein analyte in the plurality of protein analytes in each respective liquid biological sample. The training dataset is then used to train an untrained or partially untrained classifier, thus obtaining a trained classifier that provides an indication that a subject has an intracranial aneurysm, based at least in part on a plurality of abundance measures (e.g., relative protein concentrations) in one or more liquid biological samples of the subject.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “trained classifier” refers to a model (e.g., a machine learning algorithm, such as logistic regression, neural network, regression, support vector machine, clustering algorithm, decision tree etc.) with specific parameters (weights) and thresholds, ready to be applied to previously unseen samples.

As used herein, the term “untrained classifier or partially trained classifier” refers to a model (e.g., a machine learning algorithm, such as logistic regression, neural network, regression, support vector machine, clustering algorithm, decision tree etc.) with at least some unfixed parameters (weights) and thresholds, ready to be trained on a training set in order to optimize and fix the parameters and thresholds.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “subject” refers to a human (e.g., a male human, female human, fetus, pregnant female, child, or the like). In some embodiments, a subject is a male or female of any stage (e.g., a man, a women or a child).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Example System Embodiments

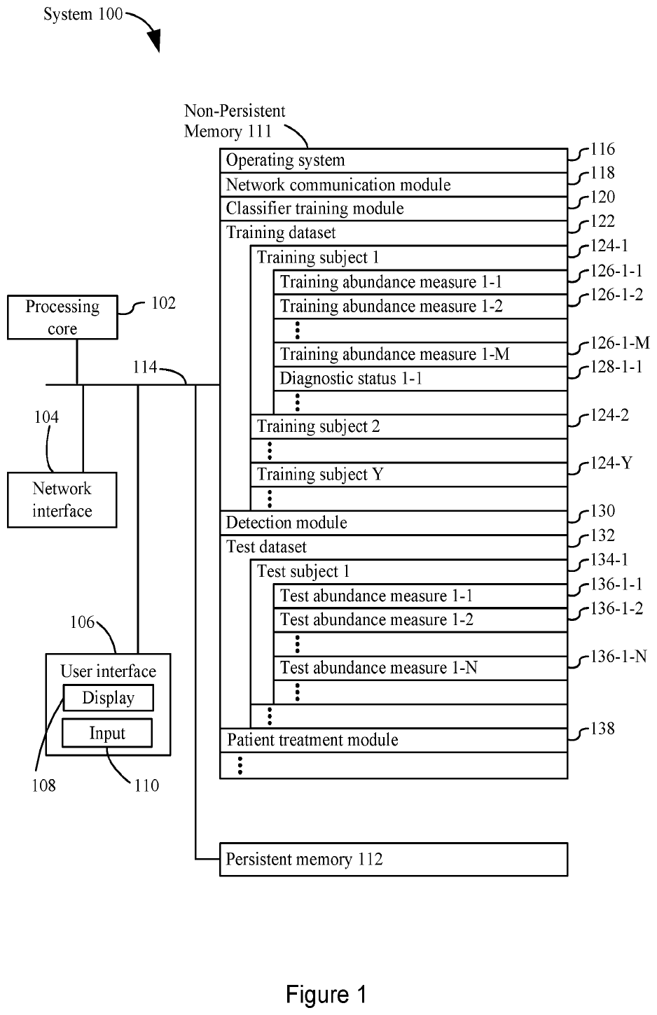

Now that an overview of some aspects of the present disclosure has been provided, details of an exemplary system are now described in conjunction with FIG. 1. FIG. 1 illustrates a block diagram of an example computing device 100, in accordance with some embodiments of the present disclosure. The device 100 in some implementations includes one or more processing units CPU(s) 102 (also referred to as processors), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 104;
a classifier training module 120 for training a classifier to provide an indication that a subject has an intracranial aneurysm;
a data store for a training dataset 122 for one or more liquid biological samples for each respective training subject 124 (e.g., 124-1, 124-2, . . . , 124-Y) in a plurality of training subjects, where each liquid biological sample comprises a plurality of protein analytes, and where the training dataset comprises, for each training subject in the plurality of training subjects, a first label indicating the corresponding first diagnostic status 128 (e.g., 128-1-1) of the respective subject and a plurality of abundance measures 126 (e.g., 126-1-1, 126-1-2, . . . , 126-1-M), each abundance measure in the plurality of abundance measures corresponding to a respective protein analyte in the plurality of protein analytes in each respective liquid biological sample in the one or more liquid biological samples;
a detection module 130 for detecting an intracranial aneurysm in a test subject, using a trained classifier;
a data store for a test dataset 132 for one or more liquid biological samples for a test subject 134 (e.g., 134-1), where each liquid biological sample comprises a plurality of protein analytes, and where the test dataset comprises a plurality of abundance measures 136 (e.g., 136-1-1, 136-1-2, . . . , 136-1-N), each abundance measure corresponding to a respective protein analyte in the plurality of protein analytes in each respective liquid biological sample in the one or more liquid biological samples; and
an optional patient treatment module 138 for determining and/or evaluating a treatment regimen or intervention for a test subject based at least in part on the indication provided by the trained classifier.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

In some embodiments, the system 100 is connected to, or includes, one or more analytical devices for performing chemical analyses. For example, the optional network communication module (or instructions) 118 is configured to connect the system 100 with the one or more analytical devices, e.g., via the communication network 104. In some embodiments, the one or more analytical devices include a mass spectrometer and/or a quantitative real-time PCR machine.

Although FIG. 1 depicts a “system 100,” the figure is intended more as functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

Detection Methods.

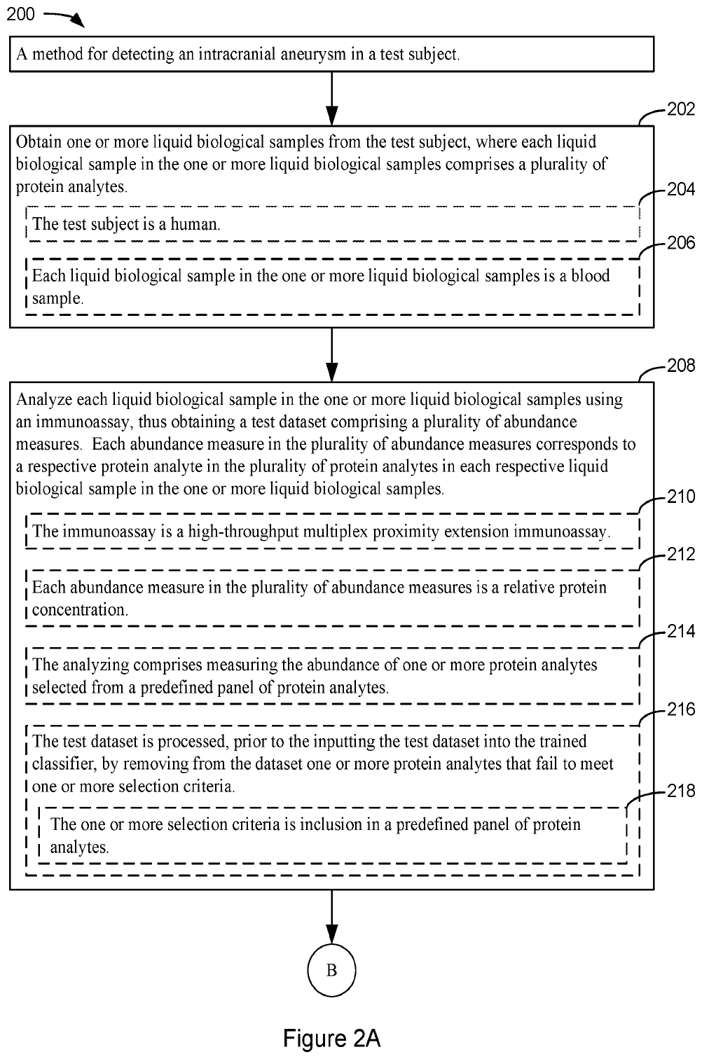

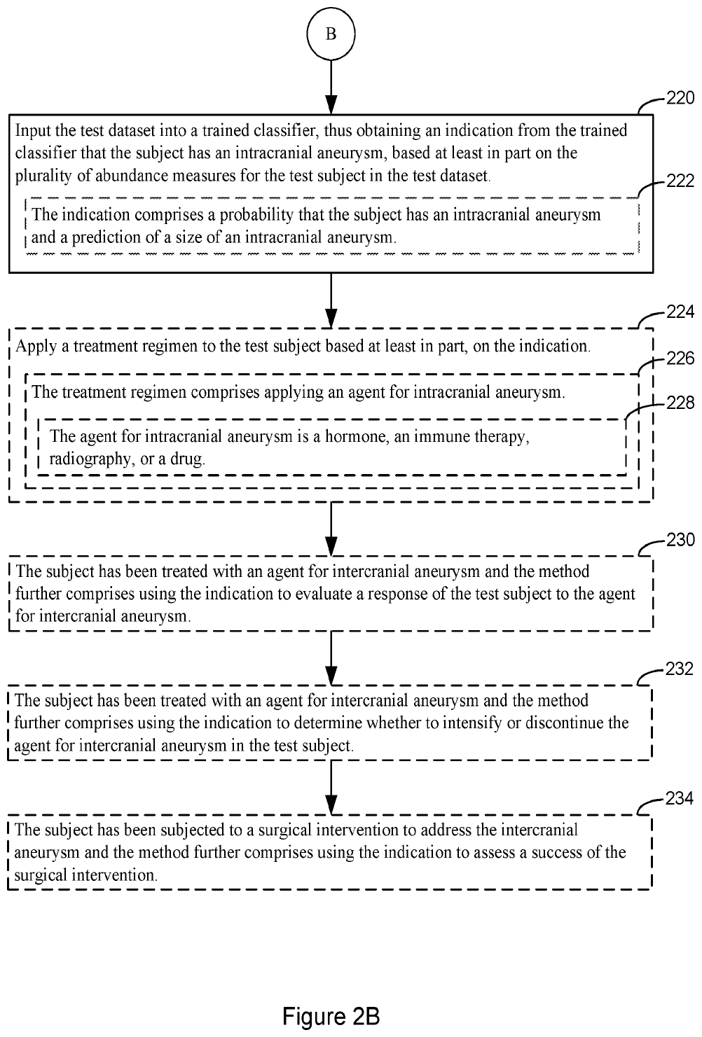

While a system in accordance with the present disclosure has been disclosed with reference to FIG. 1, detailed processes and features of a method 200 for detecting an intracranial aneurysm in a test subject, in which optional blocks are indicated with dashed boxes, in accordance with the present disclosure, is provided in conjunction with FIGS. 2A-2B.

Referring to Block 202, the method comprises obtaining one or more liquid biological samples from the test subject, where each liquid biological sample in the one or more liquid biological samples comprises a plurality of protein analytes.

Referring to Block 204, in some embodiments, the test subject is a human. For example, in some embodiments, the test subject is a patient (e.g., a study participant undergoing a diagnostic screening or a clinical evaluation). In some embodiments, the test subject has an unruptured intracranial aneurysm.

In some embodiments, one or more demographics or clinical characteristics of the test subject is collected in addition to the one or more liquid biological samples. In some embodiments, the one or more demographics or clinical characteristics comprises a respective one or more covariates, including an age of the test subject, a sex of the test subject, a hypertension status, a hyperlipidemia status, a presence or absence of diabetes mellitus type II, and/or a smoking history. In some embodiments, the test subject is a study participant, and the one or more demographics or clinical characteristics are collected prospectively through patient survey at the time of enrollment into the study. In some embodiments, the one or more demographics or clinical characteristics comprises 1, 2, 3, 4, 5, 6, 7, 8, 9, 10, or more than 10 demographics or clinical characteristics (e.g., covariates). In some embodiments, the method further comprises, in addition to the obtaining the one or more liquid biological samples, performing a diagnostic cerebral angiogram on the test subject. In some embodiments, the one or more liquid biological samples obtained from the test subject are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. Referring to Block 206, in some embodiments, each liquid biological sample in the one or more liquid biological samples is blood (e.g., whole blood, red blood cells, white blood cells, serum, and/or plasma). In some embodiments, the one or more liquid biological samples is peripheral blood. In some embodiments, blood samples are collected from patients in commercial blood collection containers. The one or more liquid biological samples can be obtained by any means known to one skilled in the art. For example, in some embodiments, the obtaining one or more liquid biological samples from the test subject is performed by venipuncture. In some embodiments, the one or more liquid biological samples from the test subject is obtained from a sample database (e.g., a pharmacogenomics biobank). In some embodiments, the liquid biological sample is separated into two different samples (e.g., by centrifugation). For example in some embodiments, a blood sample is separated into a blood plasma sample and a buffy coat preparation, containing white blood cells. In some embodiments, the separation is performed at a temperature between −10 and 20-degrees centigrade, between −5 and 15-degrees centigrade, or between 0 and 10-degrees centigrade. In some preferred embodiments, the liquid biological sample is serum. In some embodiments, each liquid biological sample in the one or more liquid biological samples has a volume of from about 1 mL to about 50 mL. For example, in some embodiments, each liquid biological sample in the one or more liquid biological samples has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater. In some embodiments, the volume of each liquid biological sample in the one or more liquid biological samples is between 0.1 μL and 1 mL.

In some embodiments, the one or more liquid biological samples is a plurality of liquid biological sample, and each liquid biological sample in the plurality of liquid biological samples is obtained from the test subject at intervals over a period of time (e.g., using serial sampling). For example, in some such embodiments, the time between obtaining liquid biological samples from a test subject is at least 1 day, at least 2 days, at least 1 week, at least 2 weeks, at least 1 month, at least 2 months, at least 3 months, at least 4 months, at least 6 months, or at least 1 year.

In some embodiments, the liquid biological sample is stored for a period of time after collection and prior to analyzing. In some such embodiments, the storage is performed at a temperature below at least 10-degrees centigrade, below at least 5-degrees centigrade, or below at least 0-degrees centigrade. In some such embodiments, the storage is performed at a temperature between −15 and −30-degrees centigrade. In some embodiments, the storage is performed at a temperature between −60 and −100-degrees centigrade. In some embodiments, the period of time is at least 1 day, at least 2 days, at least 1 week, at least 2 weeks, at least 1 month, at least 2 months, at least 3 months, at least 4 months, at least 6 months, or at least 1 year.

In some embodiments, the plurality of protein analytes comprise any peptide or polypeptide molecule contained in the liquid biological sample, including albumin, globulins, immunoglobulins, fibrinogens, circulatory proteins, secreted proteins, and/or enzymes.

Referring to Block 208, the method further comprises analyzing each liquid biological sample in the one or more liquid biological samples using an immunoassay, thus obtaining a test dataset comprising a plurality of abundance measures. Each abundance measure in the plurality of abundance measures corresponds to a respective protein analyte in the plurality of protein analytes in each respective liquid biological sample in the one or more liquid biological samples.

In some embodiments, the immunoassay is any assay capable of quantifying or detecting one or more protein analytes in the one or more liquid biological samples. For example, in some embodiments, the immunoassay is a enzyme immunoassay (EIA), a radioimmunoassay (MA), a fluoroimmunoassay (FIA), a chemiluminescent immunoassay (CLIA), a counting immunoassay (CIA), or any combination or modification thereof.

Referring to Block 210, in some embodiments, the immunoassay is a high-throughput multiplex proximity extension immunoassay. The assay is able to achieve a high level of multiplexing with robust sensitivity and specificity through the use of a “proximity extension” method, which relies on a pair of oligonucleotide-conjugated antibodies that are specific for each analyte. Upon antibody engagement with the specific analyte, the conjugated oligonucleotides are brought into close proximity, enabling their ligation and extension, as well as generation of amplicons. Relative quantification of all analytes across all patient samples can then be determined via high-throughput analysis of amplicon levels using quantitative real-time polymerized chain reactions (qRT-PCR). A high throughput proximity extension assay can also allow for the identification of a wide variety of protein analytes rather than a single protein analyte, leading to the development of a proteomic signature.

In some embodiments, the immunoassay detects one or more protein analytes in the plurality of protein analytes in each respective liquid biological sample in the one or more liquid biological samples, and provides an abundance measure for each one or more protein analytes detected. In some embodiments, the abundance measure is a concentration. In some embodiments, the abundance measure is absolute or relative. Referring to Block 212, in some embodiments, the abundance measure in the plurality of abundance measures is a relative protein concentration.

Referring to Block 214, in some embodiments, the analyzing each liquid biological sample using an immunoassay comprises measuring the abundance of one or more protein analytes selected from a predefined panel of protein analytes. In some embodiments, the predefined panel of protein analytes is an inflammatory panel (e.g., Olink Proteomics inflammatory panel). The inflammatory panel can be selected based on a priori knowledge, such as where previous biomarkers identified in IA and cerebrovascular disease are most commonly inflammatory markers or immunologic markers including adhesion molecules and complement factors. For example, in some embodiments, the predefined panel of protein analytes comprises one or more analytes selected from Table 1.

TABLE 1
Selected protein analytes for immunoassay analysis.
Protein Analyte Long Name (Short Name)

Adenosine Deaminase (ADA)
Artemin (ARTN)
Axin-1 (AXIN1)
Beta-nerve growth factor (Beta-NGF)
Caspase-8 (CASP-8)
C-C motif chemokine 3 (CCL3)
C-C motif chemokine 4 (CCL4)
C-C motif chemokine 19 (CCL19)
C-C motif chemokine 20 (CCL20)
C-C motif chemokine 23 (CCL23)
C-C motif chemokine 25 (CCL25)
C-C motif chemokine 28 (CCL28)
CD4OL receptor (CD40)
CUB domain-containing protein 1 (CDCP1)
C-X-C motif chemokine 1 (CXCL1)
C-X-C motif chemokine 5 (CXCL5)
C-X-C motif chemokine 6 (CXCL6)
C-X-C motif chemokine 9 (CXCL9)
C-X-C motif chemokine 10 (CXCL10)
C-X-C motif chemokine 11 (CXCL11)
Cystatin D (CST5)
Delta and Notch-like epidermal growth factor-related receptor (DNER)
Eotaxin (CCL11)
Eukaryotic translation initiation factor 4E-binding protein 1 (4E-BP1)
Fibroblast growth factor 21 (FGF-21)
Fibroblast growth factor 23 (FGF-23)
Fibroblast growth factor 5 (FGF-5)
Fibroblast growth factor 19 (FGF-19)
Fms-related tyrosine kinase 3 ligand (Flt3L)
Fractalkine (CX3CL1)
Glial cell line-derived neurotrophic factor (GDNF)
Hepatocyte growth factor (HGF)
Interferon gamma (IFN-gamma)
Interleukin-1 alpha (IL-1 alpha)
Interleukin-2 (IL-2)
Interleukin-2 receptor subunit beta (IL-2RB)
Interleukin-4 (IL-4)
Interleukin-5 (IL5)
Interleukin-6 (IL6)
Interleukin-7 (IL-7)
Interleukin-8 (IL-8)
Interleukin-10 (IL10)
Interleukin-10 receptor subunit alpha (IL-10RA)
Interleukin-10 receptor subunit beta (IL-10RB)
Interleukin-12 subunit beta (IL-12B)
Interleukin-13 (IL-13)
Interleukin-15 receptor subunit alpha (IL-15RA)
Interleukin-17A (IL-17A)
Interleukin-17C (IL-17C)
Interleukin-18 (IL-18)
Interleukin-18 receptor 1 (IL-18R1)
Interleukin-20 (IL-20)
Interleukin-20 receptor subunit alpha (IL-20RA)
Interleukin-22 receptor subunit alpha-1 (IL-22 RA1)
Interleukin-24 (IL-24)
Interleukin-33 (IL-33)
Latency-associated peptide transforming growth factor beta-1 (LAP
TGF-beta-1)
Leukemia inhibitory factor (LIF)
Leukemia inhibitory factor receptor (LIF-R)
Macrophage colony-stimulating factor 1 (CSF-1)
Matrix metalloproteinase-1 (MMP-1)
Matrix metalloproteinase-10 (MMP-10)
Monocyte chemotactic protein 1 (MCP-1)
Monocyte chemotactic protein 2 (MCP-2)
Monocyte chemotactic protein 3 (MCP-3)
Monocyte chemotactic protein 4 (MCP-4)
Natural killer cell receptor 2B4 (CD244)
Neurotrophin-3 (NT-3)
Neurturin (NRTN)
Oncostatin-M (OSM)
Osteoprotegerin (OPG)
Programmed cell death 1 ligand 1 (PD-L1)
Protein S100-A12 (EN-RAGE)
Signaling lymphocytic activation molecule (SLAMF1)
5IR2-like protein 2 (SIRT2)
STAM-binding protein (STAMBP)
Stem cell factor (SCF)
Sulfotransferase 1A1 (ST1A1)
T cell surface glycoprotein CD6 isoform (CD6)
T-cell surface glycoprotein CD5 (CD5)
T-cell surface glycoprotein CD8 alpha chain (CD8A)
Thymic stromal lymphopoietin (TSLP)
TNF-beta (TNFB)
TNF-related activation-induced cytokine (TRANCE)
TNF-related apoptosis-inducing ligand (TRAIL)
Transforming growth factor alpha (TGF-alpha)
Tumor necrosis factor (Ligand) superfamily, member 12 (TWEAK)
Tumor necrosis factor (TNF)
Tumor necrosis factor ligand superfamily member 14 (TNFSF14)
Tumor necrosis factor receptor superfamily member 9 (TNFRSF9)
Urokinase-type plasminogen activator (uPA)
Vascular endothelial growth factor A (VEGF-A)

In some alternative embodiments, the predefined panel includes one or more protein analytes identified, based on experimental validation or theoretical determination, as being associated with IA (e.g., a biomarker signature). For example, in some embodiments, the predefined panel of protein analytes comprises one or more analytes selected from Table 2.

TABLE 2
Selected protein analytes associated
with intracranial aneurysms.
Protein Analyte
CXCL6
CASP-8
CD40
CXCL5
CXCL1
ST1A1
EN-RAGE
Flt3L

In some embodiments, the predefined panel of protein analytes comprises one or more analytes selected from Table 4.

In some embodiments, the predefined panel of protein analytes is selected by performing a statistical analysis on a plurality of abundance measures corresponding to a plurality of protein analytes obtained from one or more training samples to identify one or more protein analytes that are correlated with IA. In some such embodiments, the statistical analysis is a univariate or a multivariate analysis.

In some embodiments, the test dataset further comprises a first label indicating a corresponding first covariate for the test subject, the indication from the trained classifier that the subject has an intracranial aneurysm is further based on the first covariate, and the corresponding first covariate is selected from the group consisting of an age of the test subject, a sex of the test subject, a hypertension status, a hyperlipidemia status, a presence or absence of diabetes mellitus type II; and/or a smoking history. For example, in some embodiments, the first covariate is a hyperlipidemia status, and the first label is “yes” or “no”. In some alternative embodiments, the first covariate is a smoking history, and the first label is selected from the group consisting of “former smoker but quit,” “current smoker,” “has not quit,” and “never smoker.” In some embodiments, the test dataset further comprises 1, 2, 3, 4, 5, 6, 7, 8, 9, 10, or more than 10 additional labels (e.g., covariates).

In some embodiments, the test dataset is pre-processed by normalization of the plurality of abundance measures prior to the inputting the test dataset into the trained classifier. In some preferred embodiments, the test dataset is processed by Z-score normalization and/or scaling (e.g., Log 2 scaling). For example, in some embodiments, the test dataset is pre-processed by normalization across all samples using a reference sample normalization method using a scaling factor between interplate controls.

Referring to Block 216, in some embodiments, the test dataset is processed, prior to the inputting the test dataset into the trained classifier, by removing from the dataset one or more protein analytes that fail to meet one or more selection criteria. In some embodiments, the one or more selection criteria is a threshold limit of detection (LOD). In some embodiments, the one or more selection criteria is a threshold variance.

Referring to Block 218, in some embodiments, the one or more selection criteria is inclusion in a predefined panel of protein analytes (e.g., Table 1, Table 2, and/or Table 4). In some such embodiments, only those abundance measures that correspond to the one or more protein analytes in the predefined panel of protein analytes are used for detecting an IA in the test subject.

More particularly, referring to Block 220, the method further comprises inputting the test dataset into a trained classifier, thus obtaining an indication from the trained classifier that the subject has an intracranial aneurysm, based at least in part on the plurality of abundance measures for the test subject in the test dataset.

In some embodiments, the trained classifier provides an indication that the subject has an intracranial aneurysm, where the indication comprises a first diagnostic status (e.g., a presence or absence of IA) and a second diagnostic status (e.g., a size of an IA, a location of an IA, a presence or absence of aneurysmal rupture, a saccular aneurysm, an endovascular treatment status for an IA, and/or an open treatment status for an IA).

In some embodiments, the indication from the trained classifier that a subject has an intracranial aneurysm further comprises 1, 2, 3, 4, 5, 6, 7, 8, 9, 10, or more than 10 additional indications, where each additional indication corresponds to a respective additional diagnostic status (e.g., a size of an IA, a location of an IA, a presence or absence of aneurysmal rupture, a saccular aneurysm, an endovascular treatment status for an IA, and/or an open treatment status for an IA) in addition to the first diagnostic status (e.g., a presence or absence of IA).

In some embodiments, the trained classifier provides an indication that the subject has an intracranial aneurysm, based at least in part on the plurality of abundance measures and one or more covariates (e.g., demographics or clinical characteristics) for the test subject in the test dataset, where the one or more covariates comprises an age of a training subject, a sex of a training subject, a hypertension status, a hyperlipidemia status, a presence or absence of diabetes mellitus type II, and/or a smoking history.

In some embodiments, the trained classifier provides an indication that the subject has an intracranial aneurysm, based at least in part on the plurality of abundance measures and 1, 2, 3, 4, 5, 6, 7, 8, 9, 10, or more than 10 additional covariates (e.g., demographics or clinical characteristics) for the test subject in the test dataset, where the 1, 2, 3, 4, 5, 6, 7, 8, 9, 10, or more than 10 additional covariates comprises an age of a training subject, a sex of a training subject, a hypertension status, a hyperlipidemia status, a presence or absence of diabetes mellitus type II, and/or a smoking history.

In some embodiments, the trained classifier can further detect any number of alternative diagnostic status and/or any combination thereof, based at least in part on the plurality of protein abundance measures and/or the plurality of protein abundance measures with any number of alternative input covariates and/or any combination thereof.

Referring to Block 222, in some preferred embodiments, the indication comprises a probability that the subject has an intracranial aneurysm and a prediction of a size of an intracranial aneurysm.

In some embodiments, the probability is provided as a number ranging from 0 to 1, where 1 corresponds to a 100% probability that the subject has an IA. In some embodiments, the indication includes applying a predetermined threshold to the obtained probability. If the obtained probability is above the predetermined threshold, the subject is evaluated as having an IA. If the obtained probability is below the predetermined threshold, the subject is evaluated as not having an IA. In some embodiments, the predetermined threshold is between 0.3-0.6 (e.g., the predetermined threshold is 0.3, 0.35, 0.4, 0.45, 0.5, 0.55, or 0.6). In some embodiments, the predetermined threshold is 0.45. In some embodiments, the obtained probability is expressed in terms of associated odds (e.g., odds ratio (OR), which may be derived from a probability such that OR=p/(1−p)). For example, the evaluation includes evaluating odds that the subject has an IA.

In some embodiments, the trained classifier is a neural network algorithm, a support vector machine algorithm, a Naïve Bayes algorithm, a decision tree algorithm, an unsupervised clustering model algorithm, a supervised clustering model algorithm, or a regression model. In some preferred embodiments, the trained classifier is a support vector machine or a multivariate logistic regression model.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as the of the multi-category logit models, are described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York.

Referring to Block 224, in some embodiments, the method further comprises applying a treatment regimen to the test subject based at least in part, on the indication. In some such embodiments, referring to Block 226, the treatment regimen comprises applying an agent for intracranial aneurysm. For example, referring to Block 228, in some embodiments, the agent for intracranial aneurysm is a hormone, an immune therapy, radiography, or a drug.

For example, treatment options for patients with intracranial aneurysms include medical (e.g., non-surgical) therapy, surgical therapy (e.g., clipping), and/or endovascular therapy (e.g., coiling).

In general, medical or non-surgical therapy is available as treatment only for unruptured intracranial aneurysms. In some cases, medical therapy is performed where the risk of preventive repair such as surgery outweighs the risk of rupture, e.g., where the size of the IA is small (e.g., 5 mm or less in diameter). Due to the role of such factors as smoking, hypertension and/or aneurysm wall inflammation in aneurysm formation, growth, and rupture, medical therapy can include a patient-modifiable strategy, such as a smoking cessation program or blood pressure control. Blood pressure control can be managed using methods including hypertensive medication and/or diet and exercise programs.

Aneurysm wall inflammation is thought to play a role in the incidence of aneurysm growth and/or rupture. As such, studies have reported that acetylsalicylic acid (ASA) can provide a protective effect against aneurysm rupture by unselectively inhibiting cyclooxygenase 2, thus decreasing aneurysm wall inflammation. As a result, agents for intracranial aneurysm can include anti-inflammatory drugs such as ASA, or other unselective or selected cyclooxygenase-2 inhibitors. See, Hackenberg et al., Stroke 49:9, 2268-2275 (2018).

Surgical therapies include clipping and endovascular coiling, both of which are designed to prevent blood flow into the aneurysm. Clipping is a surgical procedure in which the aneurysm is isolated from the surrounding brain tissue and a metal clip is applied to the base of the aneurysm. The procedure thus occludes the aneurysm, separating the aneurysm sac from cerebral circulation. Clipping presents a high risk, as the methods requires accessing the aneurysm through the skull, and careful separation of the aneurysm from the brain tissue. Endovascular coiling utilizes Guglielmi detachable coils (GDCs), or soft wire spirals that are placed inside the aneurysm by means of a microcatheter that is directed into the brain through an opening in the femoral artery of the leg. The GDCs obstruct blood flow and facilitates clotting in the aneurysm, such that the clot effectively separates the aneurysm from the cerebral circulation. Other surgical therapies include contralateral MCA aneurysm clipping, temporary artery occlusion, angiography, wrapping and clipping, bypass (e.g., intracranial-to-intracranial bypass and/or bipolar coagulating), transluminal embolization (e.g., double catheter technique, balloon-assisted coiling, stent-assisted coiling, mesh technique, Y-stenting, flow-diverting stent, salvation techniques, and/or intrasaccular flow disruptions). Many surgical techniques for IA treatment are known in the art. See, for example, Zhao et al., Angiology 69(1), 17-30 (2018).

In addition to other treatment options, radiography can be recommended as a supplemental treatment for IA as a means to monitor the size and/or growth of the aneurysm, allowing the efficacy of the treatment to be assessed over time.

Referring to Block 230, in some embodiments, the subject has been treated with an agent for intercranial aneurysm and the method further comprises using the indication to evaluate a response of the test subject to the agent for intercranial aneurysm. For example, in some such embodiments, the agent for intercranial aneurysm is a hormone, an immune therapy, radiography, or a drug.

Referring to Block 232, in some embodiments, the subject has been treated with an agent for intercranial aneurysm and the method further comprises using the indication to determine whether to intensify or discontinue the agent for intercranial aneurysm in the test subject.

Referring to Block 234, in some embodiments, the subject has been subjected to a surgical intervention to address the intercranial aneurysm and the method further comprises using the indication to assess a success of the surgical intervention.

In some such embodiments, the method comprises detecting an IA in the test subject at multiple time points over a period of time (e.g., monitoring), where the time between detection is at least 1 day, at least 2 days, at least 1 week, at least 2 weeks, at least 1 month, at least 2 months, at least 3 months, at least 4 months, at least 6 months, or at least 1 year.

In some embodiments, the method 200 described with respect to FIGS. 2A-2B is performed by a device executing one or more programs (e.g., one or more programs stored in the Non-Persistent Memory 111 or in the Persistent Memory 112 in FIG. 1) including instructions to perform the method 200. In some embodiments, the method 200 is performed by a system comprising at least one processor (e.g., the processing core 102) and memory (e.g., one or more programs stored in the Non-Persistent Memory 111 or in the Persistent Memory 112) comprising instructions to perform the method 200.

Classifier Training.

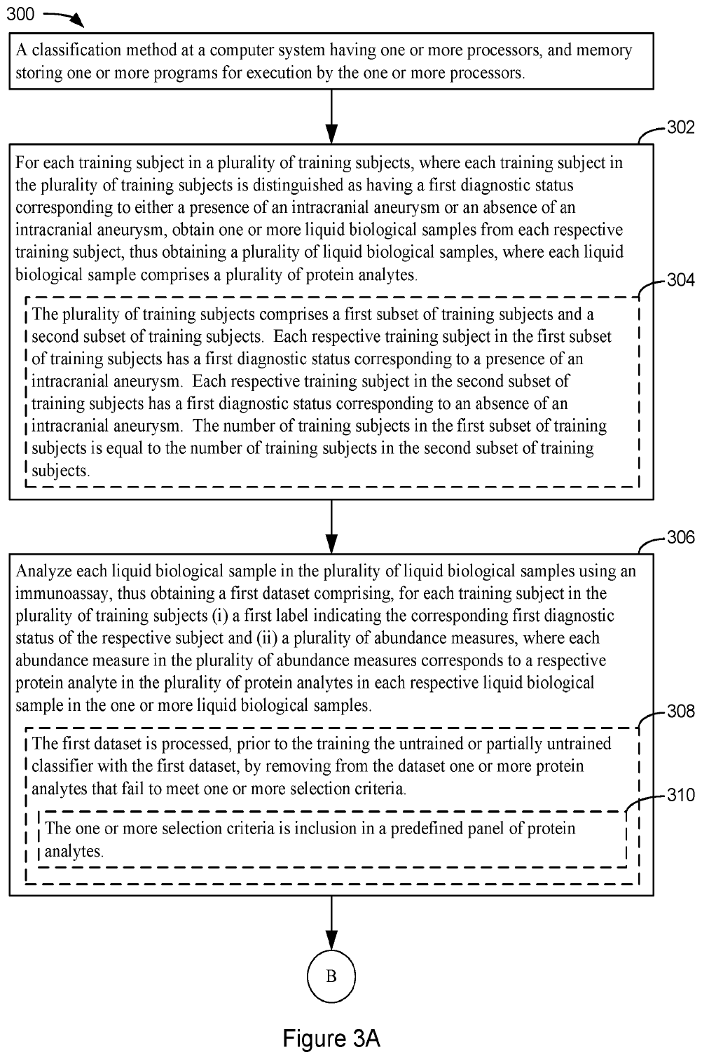

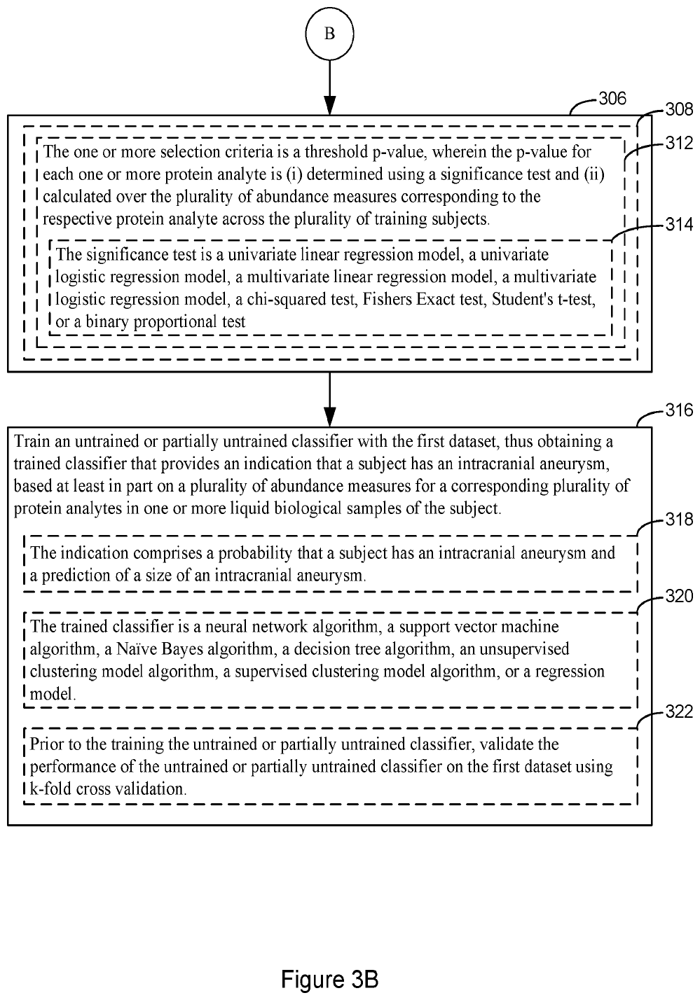

Now that the methods and features of the method 200 have been disclosed with reference to FIGS. 2A-2B, FIGS. 3A-3B provides a flow chart of processes and features of a classification method 300 for training a classifier to provide an indication that a subject has an intracranial aneurysm, in which optional blocks are indicated with dashed boxes, in accordance with some embodiments of the present disclosure.

Referring to Block 302, the method comprises, at a computer system having one or more processors, and memory storing one or more programs for execution by the one or more processors, for each training subject in a plurality of training subjects, where each training subject in the plurality of training subjects is distinguished as having a first diagnostic status corresponding to either a presence of an intracranial aneurysm or an absence of an intracranial aneurysm, obtaining one or more liquid biological samples from each respective training subject, thereby obtaining a plurality of liquid biological samples. Each liquid biological sample comprises a plurality of protein analytes.

In some embodiments, each training subject in the plurality of training subjects is a human. In some embodiments, each training subject is a patient (e.g., a study participant undergoing a diagnostic screening or a clinical evaluation). Referring to Block 304, in some embodiments, the plurality of training subjects comprises a first subset of training subjects and a second subset of training subjects, each respective training subject in the first subset of training subjects has a first diagnostic status corresponding to a presence of an intracranial aneurysm (e.g., an IA cohort), each respective training subject in the second subset of training subjects has a first diagnostic status corresponding to an absence of an intracranial aneurysm (e.g., a control cohort), and the number of training subjects in the first subset of training subjects is equal to the number of training subjects in the second subset of training subjects.

In some embodiments, one or more demographics or clinical characteristics of each training subject is collected in addition to the one or more liquid biological samples. In some embodiments, the one or more demographics or clinical characteristics comprises a respective one or more covariates, including an age of the training subject, a sex of the training subject, a hypertension status, a hyperlipidemia status, a presence or absence of diabetes mellitus type II, and/or a smoking history. In some embodiments, the training subject is a study participant, and the one or more demographics or clinical characteristics are collected prospectively through patient survey at the time of enrollment into the study.

In some embodiments, the one or more demographics or clinical characteristics of each training subject further comprises one or more inclusion criteria, including a size of an intracranial aneurysm, a location of an intracranial aneurysm, a presence or absence of aneurysmal rupture, a saccular aneurysm, an endovascular treatment status for an intracranial aneurysm, and/or an open treatment status for an intracranial aneurysm. In some embodiments, the one or more demographics or clinical characteristics comprises 1, 2, 3, 4, 5, 6, 7, 8, 9, 10, or more than 10 demographics or clinical characteristics (e.g., covariates and/or inclusion criteria).

Referring again to Block 304, in some embodiments, each respective training subject in the first subset of training subjects (e.g., the IA cohort) is matched to a respective training subject in the second subset of training subjects (e.g., the control cohort) by one or more covariates (e.g., age, sex, and/or comorbidity status).

In some alternative embodiments, the number of training subjects in the first subset of training subjects is different from the number of training subjects in the second subset of training subjects. In some embodiments, at least one respective training subject in the first subset of training subjects (e.g., the IA cohort) is not matched to a respective training subject in the second subset of training subjects (e.g., the control cohort) by one or more covariates (e.g., age, sex, and/or comorbidity status). In some embodiments, at least one respective training subject in the second subset of training subjects (e.g., the control cohort) is not matched to a respective training subject in the first subset of training subjects (e.g., the IA cohort) by one or more covariates (e.g., age, sex, and/or comorbidity status).

In some embodiments, the method further comprises, in addition to the obtaining the one or more liquid biological samples, performing a diagnostic cerebral angiogram on the training subject.

The one or more liquid biological samples obtained from each respective training subject can comprise any of the same embodiments described above for the test subject, or any substitutions or combinations thereof as will be apparent to one skilled in the art. In some embodiments, each liquid biological sample in the plurality of liquid biological samples is a blood sample.

The one or more liquid biological samples obtained from each respective training subject can be collected, processed, and/or stored using any of the same methods and/or embodiments described above for the test subject, or any substitutions or combinations thereof as will be apparent to one skilled in the art. In some embodiments, the obtaining one or more liquid biological samples from each respective training subject is performed by venipuncture.

In some embodiments, the plurality of protein analytes comprise any peptide or polypeptide molecule contained in the liquid biological sample, including albumin, globulins, immunoglobulins, fibrinogens, circulatory proteins, secreted proteins, and/or enzymes.

Referring to Block 306, the method further comprises analyzing each liquid biological sample in the plurality of liquid biological samples using an immunoassay, thereby obtaining a first dataset (e.g., a training dataset).

In some embodiments, the immunoassay is any assay capable of quantifying or detecting one or more protein analytes in the one or more liquid biological samples. For example, in some embodiments, the immunoassay is a enzyme immunoassay (EIA), a radioimmunoassay (MA), a fluoroimmunoassay (FIA), a chemiluminescent immunoassay (CLIA), a counting immunoassay (CIA), or any combination or modification thereof. In some embodiments, the immunoassay is a high-throughput multiplex proximity extension immunoassay.

In some embodiments, the immunoassay detects one or more protein analytes in the plurality of protein analytes in each respective liquid biological sample in the one or more liquid biological samples, and provides an abundance measure for each one or more protein analytes detected. In some embodiments, the abundance measure is a concentration. In some embodiments, the abundance measure is absolute or relative. For example, in some embodiments, the abundance measure in the plurality of abundance measures is a relative protein concentration.

Referring again to Block 306, the first dataset comprises, for each training subject in the plurality of training subjects, a first label indicating the corresponding first diagnostic status (e.g., a presence or absence of IA) of the respective subject. In some embodiments, the first dataset further comprises, for each subject in the plurality of subjects, a second label indicating a corresponding second diagnostic status, wherein the second diagnostic status is selected from the group consisting of a size of an intracranial aneurysm, a location of an intracranial aneurysm, a presence or absence of aneurysmal rupture, a saccular aneurysm, an endovascular treatment status for an intracranial aneurysm, an open treatment status for an intracranial aneurysm, an age of a training subject, a sex of a training subject, a hypertension status, a hyperlipidemia status, a presence or absence of diabetes mellitus type II and/or a smoking history. Thus, in some embodiments, the corresponding second diagnostic status is any of the one or more demographics or clinical characteristics (e.g., the one or more covariates and/or one or more inclusion criteria) obtained from each training subject in the plurality of training subjects.

In some embodiments, the first dataset further comprises, for each training subject in the plurality of training subjects, 1, 2, 3, 4, 5, 6, 7, 8, 9, 10, or more than 10 additional labels (e.g., covariates and/or inclusion criteria).

Referring again to Block 306, the first dataset further comprises, for each training subject in the plurality of training subjects, a plurality of abundance measures, where each abundance measure in the plurality of abundance measures corresponds to a respective protein analyte in the plurality of protein analytes in each respective liquid biological sample in the one or more liquid biological samples.

In some embodiments, the analyzing each liquid biological sample using an immunoassay comprises measuring the abundance of one or more protein analytes selected from a predefined panel of protein analytes. In some such embodiments, the predefined panel of protein analytes comprises one or more analytes selected from Table 1. In some alternative embodiments, the predefined panel of protein analytes comprises one or more analytes selected from Table 2. In some embodiments, the predefined panel of protein analytes comprises one or more analytes selected from Table 4.

In some embodiments, the first dataset is pre-processed by normalization of the plurality of abundance measures prior to the training the untrained or partially untrained classifier with the first dataset. For example, the first dataset (e.g., the training dataset), can be pre-processed using any of same the methods and/or embodiments of pre-processing a test dataset, described above.

Referring to Block 308, in some embodiments, the first dataset is processed, prior to the training the untrained or partially untrained classifier with the first dataset, by removing from the dataset one or more protein analytes that fail to meet one or more selection criteria. In some embodiments, the one or more selection criteria is a threshold limit of detection.

Referring to Block 310, in some embodiments, the one or more selection criteria is inclusion in a predefined panel of protein analytes (e.g., Table 1, Table 2, and/or Table 4). In some such embodiments, only those abundance measures that correspond to the one or more protein analytes in the predefined panel of protein analytes are used for training a classifier to provide an indication of an IA in a subject.

Referring to Block 312, in some embodiments, the one or more selection criteria is a threshold p-value, wherein the p-value for each one or more protein analyte is (i) determined using a significance test and (ii) calculated over the plurality of abundance measures corresponding to the respective protein analyte across the plurality of training subjects.

In some embodiments, the calculated p-value indicates the significance of correlation of an abundance measure corresponding to a respective protein analyte to the corresponding first diagnostic status (e.g., the correlation of an enrichment or a depletion of a protein analyte to a presence or an absence of IA), calculated over the plurality of abundance measures corresponding to the respective protein analyte across the plurality of training subjects (e.g., across the IA cohort and the control cohort).

In some embodiments, the calculated p-value indicates the degree of enrichment of one or more abundance measures, each abundance measure corresponding to a respective protein analyte, calculated over the plurality of abundance measures corresponding to a plurality of protein analytes (e.g., the enrichment or depletion of one or more protein analytes compared to all other protein analytes in a sample).

In some embodiments, the calculated p-value indicates the significance of correlation of an abundance measure corresponding to a respective protein analyte to a corresponding second diagnostic status (e.g., a size of an intracranial aneurysm, a location of an intracranial aneurysm, a presence or absence of aneurysmal rupture, a saccular aneurysm, an endovascular treatment status for an intracranial aneurysm, an open treatment status for an intracranial aneurysm, an age of a training subject, a sex of a training subject, a hypertension status, a hyperlipidemia status, a presence or absence of diabetes mellitus type II and/or a smoking history). In some such embodiments, the p-value is calculated over the plurality of abundance measures corresponding to the respective protein analyte across the plurality of training subjects (e.g., across the IA cohort and the control cohort). In some embodiments, the p-value is calculated over the plurality of abundance measures corresponding to the plurality of protein analytes in each respective liquid biological sample in the plurality of liquid biological samples.

In some embodiments, the identification of each one or more protein analyte that meets the threshold p-value is determined prior to the removing from the dataset one or more protein analytes that fail to meet one or more selection criteria. For example, in some such embodiments, the identification of each one or more protein analyte that meets the threshold p-value is determined using a first training dataset that is used to identify the predefined panel of protein analytes, and the removing from the dataset one or more protein analytes that fail to meet one or more selection criteria is performed using a second, subsequent training dataset that is used to train the untrained or partially untrained classifier.

Referring to Block 314, in some embodiments, the significance test is a univariate linear regression model, a univariate logistic regression model, a multivariate linear regression model, a multivariate logistic regression model, a chi-squared test, Fishers Exact test, Student's t-test, or a binary proportional test.

In some embodiments, the threshold p-value is 0.05. In some embodiments, the threshold p-value is 0.0001.

Referring to Block 316, the method further comprises training an untrained or partially untrained classifier with the first dataset, thus obtaining a trained classifier that provides an indication that a subject has an intracranial aneurysm, based at least in part on a plurality of abundance measures for a corresponding plurality of protein analytes in one or more liquid biological samples of the subject.

In some embodiments, the first dataset further comprises, for each subject in the plurality of subjects, a second label indicating a corresponding second diagnostic status, and the indication from the trained classifier that a subject has an intracranial aneurysm is further based on the second diagnostic status (e.g., a size of an intracranial aneurysm, a location of an intracranial aneurysm, a presence or absence of aneurysmal rupture, a saccular aneurysm, an endovascular treatment status for an intracranial aneurysm, an open treatment status for an intracranial aneurysm, an age of a training subject, a sex of a training subject, a hypertension status, a hyperlipidemia status, a presence or absence of diabetes mellitus type II and/or a smoking history).

In some such embodiments, the indication from the trained classifier that a subject has an intracranial aneurysm is further based on 1, 2, 3, 4, 5, 6, 7, 8, 9, 10, or more than 10 additional diagnostic status.

In some embodiments, the indication further comprises an indication that the subject has the second diagnostic status (e.g., a size of an IA, a location of an IA, a presence or absence of aneurysmal rupture, a saccular aneurysm, an endovascular treatment status for an IA, and/or an open treatment status for an IA).

In some embodiments, the indication further comprises 1, 2, 3, 4, 5, 6, 7, 8, 9, 10, or more than 10 additional indications, where each additional indication corresponds to a respective additional diagnostic status (e.g., a size of an IA, a location of an IA, a presence or absence of aneurysmal rupture, a saccular aneurysm, an endovascular treatment status for an IA, and/or an open treatment status for an IA) in addition to the first diagnostic status (e.g., a presence or absence of IA).

Referring to Block 318, in some preferred embodiments, the indication comprises a probability that the subject has an intracranial aneurysm and a prediction of a size of an intracranial aneurysm. In some embodiments, the probability is provided as a number ranging from 0 to 1, where 1 corresponds to a 100% probability that the subject has an IA. In some embodiments, the indication includes applying a predetermined threshold to the obtained probability. If the obtained probability is above the predetermined threshold, the subject is evaluated as having an IA. If the obtained probability is below the predetermined threshold, the subject is evaluated as not having an IA. In some embodiments, the predetermined threshold is between 0.3-0.6 (e.g., the predetermined threshold is 0.3, 0.35, 0.4, 0.45, 0.5, 0.55, or 0.6). In some embodiments, the predetermined threshold is 0.45. In some embodiments, the obtained probability is expressed in terms of associated odds (e.g., odds ratio (OR), which may be derived from a probability such that OR=p/(1−p)). For example, the evaluation includes evaluating odds that the subject has an IA.

In some embodiments, the trained classifier can further detect any number of alternative diagnostic status and/or any combination thereof, based at least in part on the plurality of protein abundance measures and/or the plurality of protein abundance measures with any number of alternative input covariates and/or any combination thereof.

Referring to Block 320, in some embodiments, the trained classifier is a neural network algorithm, a support vector machine algorithm, a Naïve Bayes algorithm, a decision tree algorithm, an unsupervised clustering model algorithm, a supervised clustering model algorithm, or a regression model. For example, the classifier can comprise any of the same embodiments described herein, or any substitutions or combinations thereof as will be apparent to one skilled in the art.

In some embodiments, the untrained or partially untrained classifier is associated with a plurality of weights, and training the untrained or partially untrained classifier with the first dataset comprises updating the plurality of weights, thus obtaining the trained classifier, where the trained classifier is associated with an updated plurality of weights. In some embodiments, the updating of the plurality of weights is performed using backpropagation. For example, in some simplified embodiments of machine learning (e.g., deep learning), backpropagation is a method of training a network with hidden layers comprising a plurality of weights. The output of the untrained or partially untrained classifier using the initial weights (e.g., the classification of the first diagnostic status in accordance with the plurality of weights) is compared with the actual classification (e.g., the first diagnostic status corresponding to a presence or an absence of an IA) and the error is computed (e.g., using a loss function). The weight values are then updated such that the error is minimized (e.g., according to the loss function). In some embodiments, any one of a variety of backpropagation algorithms and/or methods are used to update the first and second plurality of weights, as will be apparent to one skilled in the art.

In some embodiments, training the untrained or partially untrained classifier forms a trained classifier following a first evaluation of an error function. In some such embodiments, training the untrained or partially untrained classifier forms a trained classifier following a first updating of one or more weights based on a first evaluation of an error function. In some alternative embodiments, training the untrained or partially untrained classifier forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, training the untrained or partially untrained classifier forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more weights based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, training the untrained or partially untrained classifier forms a trained classifier when the trained classifier satisfies a minimum performance requirement. For example, in some embodiments, training the untrained or partially untrained classifier forms a trained classifier when the error calculated for the trained classifier, following an evaluation of an error function across the first dataset satisfies an error threshold. In some embodiments, the error calculated by the error function across the first dataset satisfies an error threshold when the error is less than 20 percent, less than 18 percent, less than 15 percent, less than 10 percent, less than 5 percent, or less than 3 percent.

In some embodiments, training the untrained or partially untrained classifier forms a trained classifier when the classifier satisfies a minimum performance requirement based on a validation training.

For example, referring to Block 322, in some embodiments, prior to the training the untrained or partially untrained classifier, the performance of the untrained or partially untrained classifier is validated on the first dataset using k-fold cross validation.

In some such embodiments, the first dataset (e.g., the training dataset) is divided into K bins. For each fold of training, one bin in the plurality of K bins is left out of the training dataset and the classifier is trained on the remaining K−1 bins. Performance of the trained classifier is then evaluated on the K^{th} bin that was removed from the training. This process is repeated K times, until each bin has been used once for validation. In some embodiments, K is 1, 2, 3, 4, 5, 6, 7, 8, 9, 10, 11, 12, 13, 14, 15, 16, 17, 18, 19, 20, or more than 20. In some embodiments, K is between 2 and 60. In some embodiments, K is 5. In some embodiments, K is 50. In some embodiments, K=N, where N is the number of unique protein analytes in the first dataset.

In some embodiments, validation is performed using K-fold cross-validation with shuffling. In some such embodiments, K-fold cross-validation is repeated by shuffling the training dataset and performing a second K-fold cross-validation training. The shuffling is performed so that each bin in the plurality of K bins in the second K-fold cross-validation is populated with a different (e.g., shuffled) subset of training data. In some such embodiments, the validation comprises shuffling the training dataset 1, 2, 3, 4, 5, 6, 7, 8, 9, 10, or more than 10 times.

In some embodiments, K-fold cross-validation is further used to select and/or optimize parameters and/or hyperparameters (e.g., learning rate, penalties, etc.) for the trained classifier. In some embodiments, hyperparameters are predetermined and/or selected by a user or practitioner.

In some embodiments, training is performed on a plurality of machines (e.g., computers and/or systems).

In some embodiments, training the untrained or partially untrained classifier further comprises fixing one or more weights in the plurality of weights, thereby obtaining a corresponding trained classifier that can be used to perform classification (e.g., an indication of a first diagnostic status).

Other parameters and architectures can be used for training as will be apparent to one skilled in the art.

In some embodiments, the method 300 described with respect to FIG. 3A-3B is performed by a device executing one or more programs (e.g., one or more programs stored in the Non-Persistent Memory 111 or in the Persistent Memory 112 in FIG. 1) including instructions to perform the method 300. In some embodiments, the method 300 is performed by a system comprising at least one processor (e.g., the processing core 102) and memory (e.g., one or more programs stored in the Non-Persistent Memory 111 or in the Persistent Memory 112) comprising instructions to perform the method 300.

Another aspect of the present disclosure provides a device for detecting an intracranial aneurysm in a test subject, comprising one or more processors, and memory storing one or more programs for execution by the one or more processors. Another aspect of the present disclosure provides a device for a classification method, comprising one or more processors, and memory storing one or more programs for execution by the one or more processors.

In some embodiments, the one or more programs comprise instructions for performing any of the methods and embodiments described herein and/or any combinations or alternatives thereof as will be apparent to one skilled in the art.

Another aspect of the present disclosure provides a non-transitory computer readable storage medium and one or more computer programs embedded therein, the one or more computer programs comprising instructions which, when executed by a computer system, cause the computer system to perform a method for detecting an intracranial aneurysm in a test subject. Another aspect of the present disclosure provides a non-transitory computer readable storage medium and one or more computer programs embedded therein, the one or more computer programs comprising instructions which, when executed by a computer system, cause the computer system to perform a method for classification.

In some embodiments, the one or more computer programs cause the processor to perform any of the methods and embodiments described herein and/or any combinations or alternatives thereof as will be apparent to one skilled in the art.

### EXAMPLES

### Example 1: Selection of Protein Analytes associated with Intracranial Aneurysms

In an example study, proteomic data from patients with known intracranial aneurysms and age, sex and comorbidity matched controls were utilized to identify a proteomic signature that was highly consistent with the presence of an intracranial aneurysm.

Subject Demographics and Clinical Characteristics

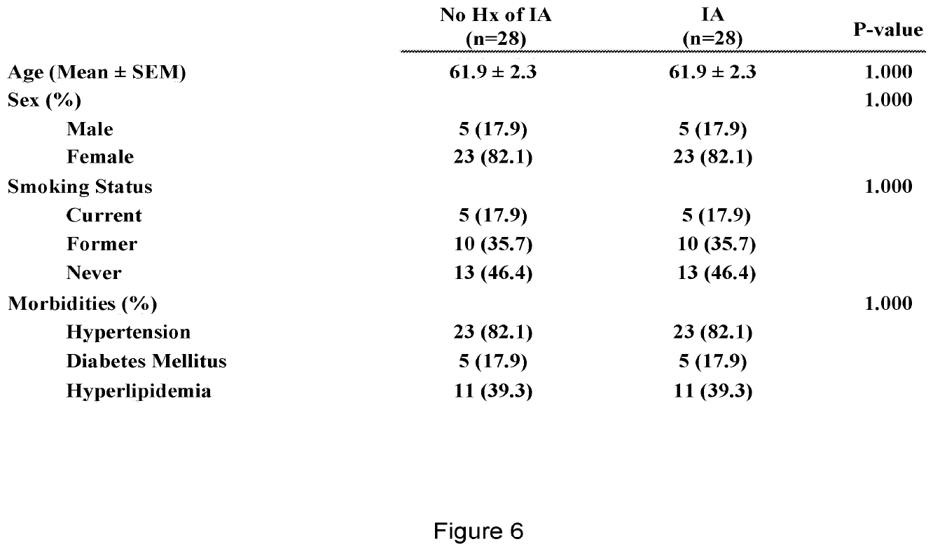

The demographics and clinical characteristics of the study participants are presented in FIG. 6 and Table 3 (IA=Intracranial Aneurysm; SEM=Standard Error of the Mean; P<0.05 was used as a threshold for statistical significance). During the study, 56 blood samples were collected: 28 from patients with IA and 28 from control subjects perfectly matched on the basis of age (p=1.000, Student's t-test), sex (p=1.000, x2 test), and comorbidities (p=1.000, x2 test). In both cohorts, 82.1% (n=23) of the patients were female. History of hypertension, diabetes mellitus, and hyperlipidemia was present for 82.1%, 17.9%, and 39.3%, respectively. A history of smoking was present for 53.6% (n=15) of the patients. The mean intracranial aneurysm size was 8.9 mm with the most common location being the anterior communicating artery (35.7%, n=10).

TABLE 3
Demographics of the study population by history of intracranial aneurysm.

| | Intracranial Aneurysm | Control | P-value |
|---|---|---|---|
| Age (Mean ± SD) | 61.9 ± 12.2 | 61.9 ± 12.2 | NS |
| Sex (%) | | | NS |
| Male | 5 (17.8) | 5 (17.8) | |
| Female | 23 (82.1) | 23 (82.1) | |
| Aneurysm Size (mm) | | | |
| Mean | 4.2 (1.6) | NA | |
| Range | 3.8 − 22.6 | NA | |

Subject Selection, Enrollment, and Clinical Data Collection

All patients with intracranial aneurysms were prospectively enrolled and informed consent was obtained. Subject inclusion criteria for the IA cohort included; (1) the presence of an intracranial aneurysm, (2) no evidence of aneurysmal rupture, (3) saccular aneurysm, and (4) no history of endovascular or open treatment of IA. All subjects with IA underwent a diagnostic cerebral angiogram and serum was collected just prior to the start of the procedure. Clinical data collection for the IA patient cohort was collected through patient interview and clinical chart review when subjects were not available for interview.

Control subjects were enrolled retrospectively utilizing an institutional pharmacogenomic biobank with over 25,000 patients enrolled with both clinical data and genetic material available for research purposes. Clinical data were collected prospectively through patient survey and International Classification of Disease, Ninth and Tenth Revision, Clinical Modification (ICD-9-CM and ICD-10-CM) codes at the time of their enrollment into the biobank. Control subjects were 1:1 matched to IA subjects by age, sex, and comorbidity status. Comorbidities included hypertension, hyperlipidemia, diabetes mellitus type II (present or not present), and smoking history (defined as current smoker, previous smoker, or never smoker).

Sample Collection and Biofluid Processing

For plasma collection, subjects with aneurysms had whole blood drawn immediately prior to their cerebral diagnostic angiogram procedure (with eight or more hours of fasting) by venipuncture. Blood samples were then centrifuged at 4-degrees centigrade. Plasma was isolated and stored for proteomic analysis. Plasma from control subjects was isolated and stored at −80-degrees centigrade per BioMe™ protocol. Plasma was prepared per Olink Proteomics (Olink Proteomics, Uppsala, Sweden) for high throughput multiplex immunoassay analysis. Methods for plasma separation and high throughput multiplex immunoassay analysis are known in the art and are described, for example, in Enroth et al., EBioMedicine 12, 309-314 (2016); and Assarsson et al., PLoS One 9, e95192 (2014).

High Throughput Multiplex Immunoassay Analysis

Olink Proteomics inflammatory panel (see, e.g., Table 1) was selected for biomarker discovery. The assay is able to achieve a high level of multiplexing with robust sensitivity and specificity through the use of a “proximity extension” method, which relies on a pair of oligonucleotide-conjugated antibodies that are specific for each analyte. Upon antibody engagement with the specific analyte, the conjugated oligonucleotides are brought into close proximity, enabling their ligation and extension, as well as generation of amplicons. Relative quantification of all analytes across all patient samples may then be determined via high-throughput analysis of amplicon levels using quantitative real-time polymerized chain reactions (qRT-PCR). The inflammatory panel was selected given that previous biomarkers identified in IA and cerebrovascular disease are most commonly inflammatory markers or immunologic markers including adhesion molecules and complement factors. A high throughput proximity extension assay such as Olink also allows for the identification of a wide variety of biomarkers lending to the development of a proteomic signature rather than identifying a single protein.

Preprocessing and Normalization of Proteomic Data

Protein concentrations underwent pre-processing normalization using a Log 2 scale allowing for relative protein concentration comparison. Samples processed on separate plates were normalized across the population using a reference sample normalization method where a scaling factor was created between interplate controls processed on both assay runs (See, e.g., Hammarskjolds, “Data normalization and standardization,” Olink Proteomics, 2018). Interplate controls after reference normalization with a scaling factor reached extremely high rates of intra-sample similarity indicating successful normalization across plates (AUC: 0.99).

Statistical Analysis

Preliminary components analysis revealed 1 of 92 analytes had zero detectability (BDNF). Z-score normalization was then performed across the remaining 91 analytes to determine variability amongst the total subject population. Samples with low variance across all subjects were removed from analysis. Given that all clinical covariates of interest were matched on a 1:1 basis between IA subjects and controls, covariates were not included in univariate or multivariate analysis or considered for signature development. Univariate logistic regression analysis was performed to identify which proteins independently correlated with the presence of IA. Binary proportional testing was utilized for signature development. Categorical variables were analyzed using chi-squared and Fisher's Exact tests and continuous variables were analyzed using Student's t-tests. Multivariate analyses included logistic regression analysis, binary proportion testing, and support vector machine (SVM) learning algorithm analysis. Normalization procedures, variance calculations, and SVM were performed using the Python data analysis library, pandas, sklearn, and Clustergrammer (Python Software Foundation. Python Language Reference, version 3.7). All other analyses were performed using R 3.5.1 (R Foundation for Statistical Computing, Vienna, Austria). See, for example, Fernandez et al., Sci Data 4, 170151 (2017); and Ashton et al., Sci Adv 5, eaau7220 (2019).

Plasma Protein Characteristics

Proteomic data derived from the inflammatory panel of the multiplex immunoassay for 28 patients with IA and 28 control subjects was utilized for data analysis. After Z-score normalization, 20 proteins were eliminated from the 92 protein analytes in the Olink inflammatory panel due to low variance across conditions and for not meeting the limit of detection (LOD) for the immunoassay. 72 analytes were differentially expressed across the subject population and used for linear and logistic regression modeling as well as SVM modeling.

Plasma Proteins Associated with Increased Aneurysm Size

Multivariate and univariate linear regression models were constructed using biological and statistical inferences to predict aneurysm size. Upon univariate analysis, we found that a number of factors were independently predictive of aneurysm size. Clinical characteristics included age, a former smoking status, and aneurysm neck size. Age (p=0.015), former smoking status (p=0.008), and the expressions of 12 inflammatory markers were significantly associated with aneurysm size at the univariate level (Table 4).

TABLE 4
Analytes predictive of large intracranial aneurysm size.

| Independent Variables | P-value |
|---|---|
| Age | 0.015 |
| Neck Size | 0.0014 |
| Former Smoker | 0.0080 |
| IL6 | 0.00070 |
| CXCL9 | 0.0012 |
| OSM | 0.013 |
| TGF Alpha | 0.013 |
| FGF21 | 0.016 |
| MMP10 | 0.019 |
| CD5 | 0.047 |
| CCL3 | 0.044 |
| CXCL10 | 0.0019 |
| EN-RAGE | 0.036 |
| CCL20 | 0.0043 |
| CSF1 | 0.019 |

The multivariate linear regression model was created using variables that were determined to be clinically important based on the literature. Former smoking status remained significant after controlling for all other variables. Additionally, IL-6 and CCL20 remained significant after control for covariates. The optimized multivariate model constructed with 6 variables was highly predictive of aneurysm size (adj R²=0.57, RMSE=3.03, F-statistic: p=0.00039). In this model, former smoking status (p=0.038), IL-6 (p=0.011), and CCL20 (p=0.025) expressions remained significant, while age (p=0.056), CCL3 (p=0.234), and EN-RAGE (p=0.184) expressions did not.

Validation of Machine Learning Algorithms for IA Prediction and Classification

Machine learning and regression algorithms were developed with an 80/20 training/test data separation to determine the precision and reliability of the proteomic signature, using the 72 analytes selected for SVM analysis after Z-score normalization.

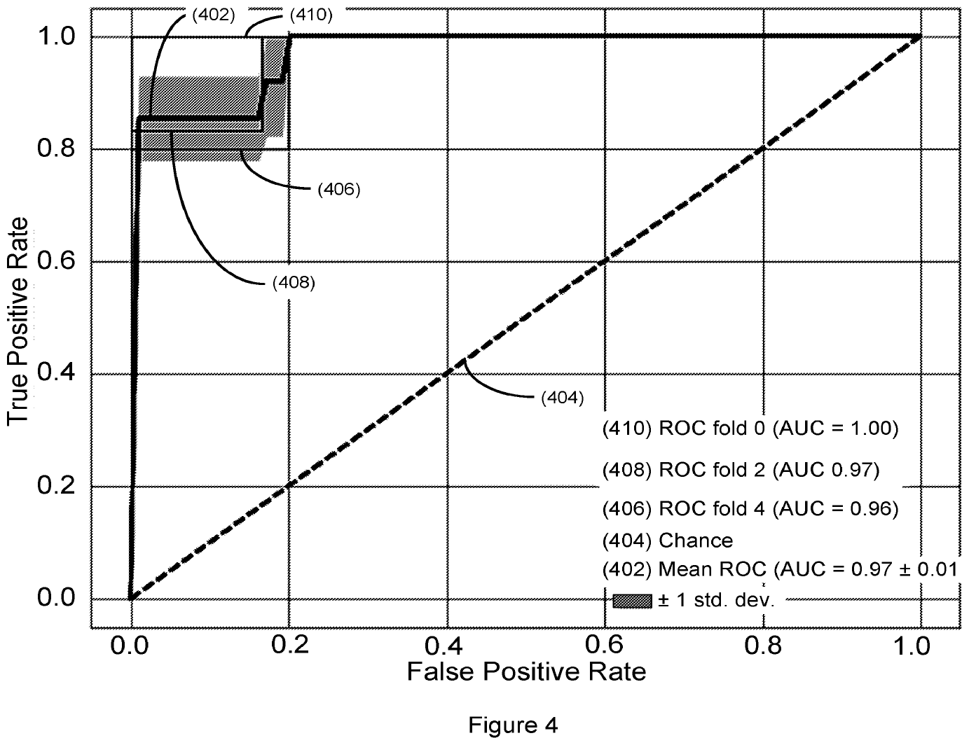

Prior to model training, 5-fold cross validation was performed at a training and test split of 80:20 percent of the study population. Each training set consisted of a random assimilation of 44 subjects from both the IA cohort and the control cohort. Each test set included the remaining 12 subjects. Receiver operating characteristic (ROC) curves were generated for each individual K-fold as well as a mean ROC curve over all 5-fold cross validations (FIG. 4, where 3 such validations are illustrated as well as the mean ROC for all five cross validations). The mean area under the curve (AUC) for the detection of intracranial aneurysms using SVM modeling was 0.97±0.01. Classification accuracy was determined using a confusion matrix which revealed a sensitivity of 1.0, a specificity of 0.83 and an F−1 score of 0.92 (Table 5).

In Table 5, “Precision” indicates the Positive Predictive Value (PPV); “Recall” indicates the Sensitivity or the True Positive Rate (TPR); “F1-Score” is the harmonic mean of precision and recall; and “Support” represents the number of subjects in each group. There were a total of 12 subjects in the test group represented by the confusion matrix in Table 5. P<0.05 was used as a threshold for statistical significance.

TABLE 5
Confusion matrix of text subjects from the Support Vector Machine (SVM)
algorithm.

| | Precision | Recall | F1-Score | Support |
|---|---|---|---|---|
| IA Absent (0) | 1.0 | 0.83 | 0.91 | 6 |
| IA Present (1) | 0.86 | 1.00 | 0.92 | 6 |
| Micro Avg. | 0.92 | 0.92 | 0.92 | 12 |
| Macro Avg. | 0.93 | 0.92 | 0.92 | 12 |
| Weighted Avg. | 0.93 | 0.92 | 0.92 | 12 |

In addition to the SVM analysis, a naïve Bayes classification algorithm was also utilized to validate the performance of IA classification based on the proteomic signature. Both the SVM and naïve Bayes classification algorithms performed well with a positive predictive value of 100% and 85.7% and a sensitivity of 100% and 100%, respectively (Brier score=0.032, 0.083).

Signature Development

Support vector machine (SVM) modeling was utilized to determine the precision and accuracy in which the proteomic expression across the two study populations could be used to classify patients into either group (see, e.g., Ashton et al., Sci Adv 5, eaau7220, 2019). To determine which analytes were associated with the presence of IA's, a binomial proportions test was performed.

The null hypothesis for the binary proportion testing was that there was an equal proportion of proteomic expression in each subject cohort. A significance threshold of p<0.0001 was used for binary proportion testing in order to determine which analytes were most significantly driving the classification of subjects. The analytes that met the significance threshold were selected for signature development.

Logistic regression analysis revealed eight highly sensitive analytes that met the significance threshold for signature development and were thus predictive of the presence of an aneurysm at a threshold of p<0.0001. The eight protein analytes identified in the biomarker signature are listed in Table 2. Seven of the analytes had proportionally higher expression in patients with IAs, where as one analyte, Flt3L, had proportionally decreased expression.

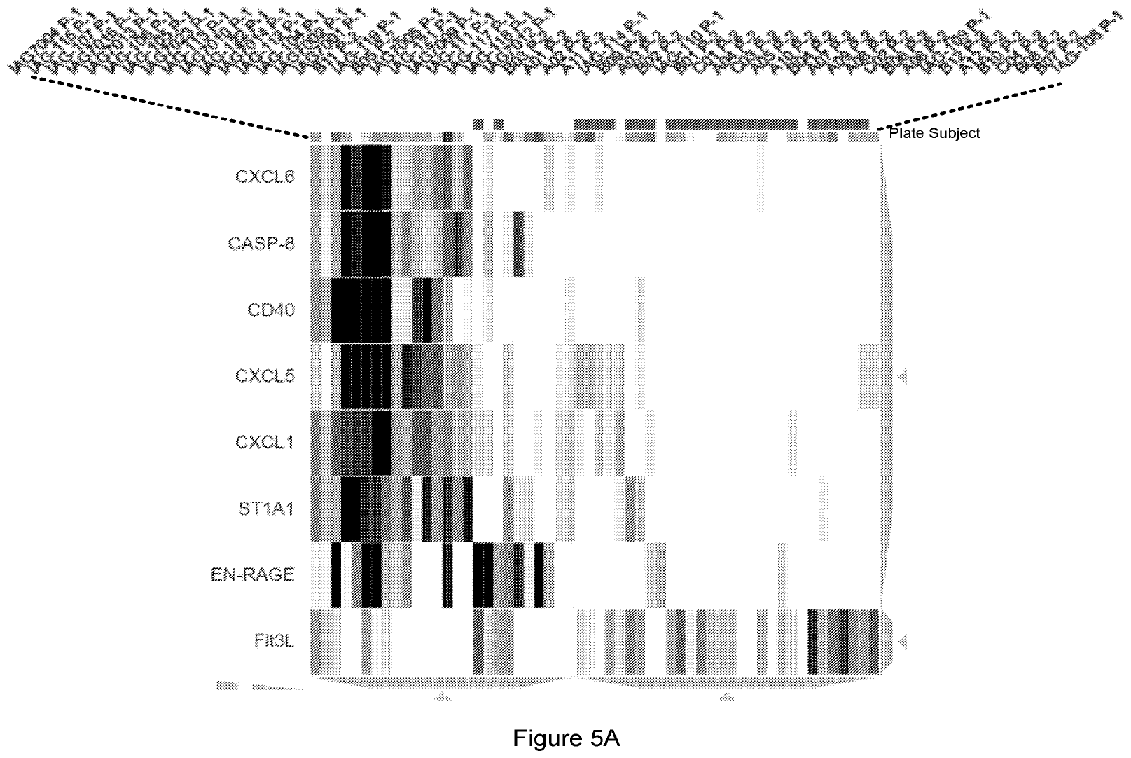

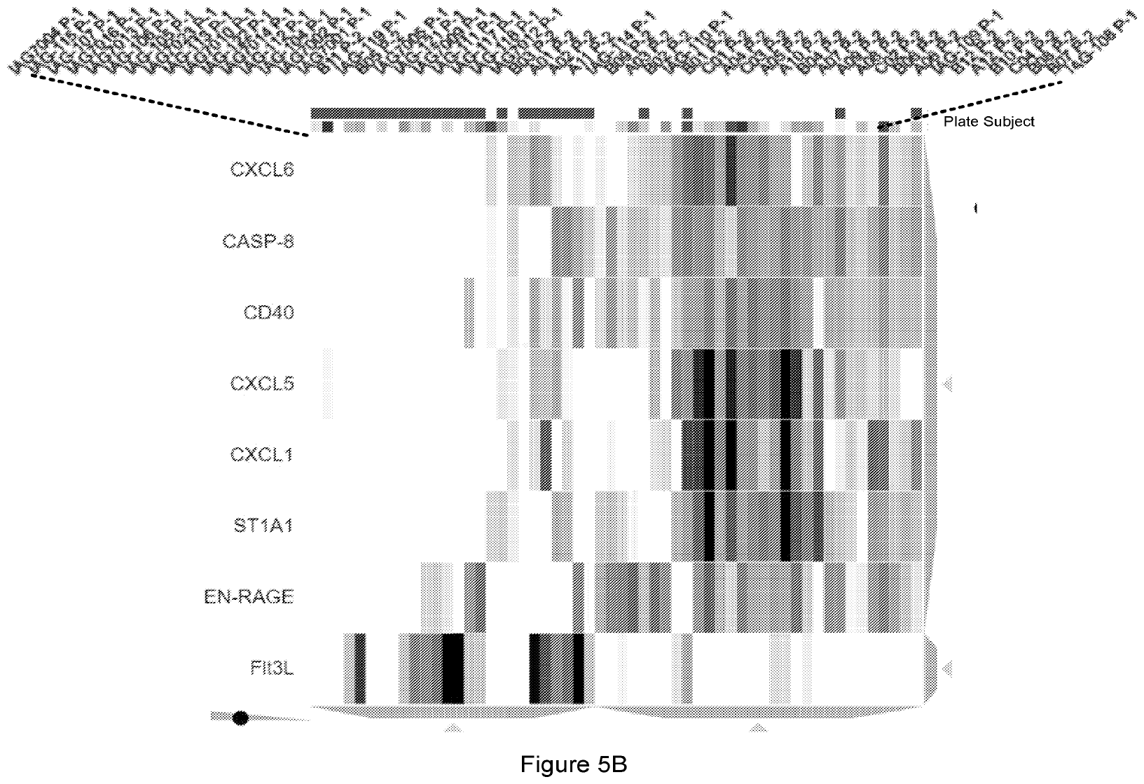

FIG. 5 illustrates the relative abundance of the eight protein analytes in IA samples compared with control samples (“Plate”: purple markers indicate IA samples, while orange markers indicate control samples). Individual patient samples are indicated by a unique color marker under “Subject.” Relative abundance of the protein analytes are indicated as upregulated/enriched (shaded blocks in FIG. 5A) or downregulated/depleted (shaded blocks in FIG. 5B), where the intensity of the shading indicates the degree of enrichment or depletion respectively.

Prediction of IAs using Proteomic Signature

Multivariate logistic regression analysis was used to determine the odds of having an IA given the proteomic expression of each analyte while controlling for relative expression of other proteins. Table 6 provides the odds ratios for each of the eight identified proteins in the proteomic signature. The odds ratio is a statistic that quantifies the degree of association between two conditions or events. An odds ratio greater than 1 indicates a positive association (e.g., a positive correlation) between the two conditions, while an odds ratio less than 1 indicates a negative association (e.g., a negative correlation). An odds ratio of 1 indicates that the two conditions are independent. In Table 6, CI=Confidence Interval; IA=Intracranial Aneurysm; OR=Odds Ratio; SEM=Standard Error of the Mean. P<0.05 was used as a threshold for statistical significance.

TABLE 6
Odds ratio and confidence intervals for likelihood of
analytes to predict presence of an intracranial aneurysm.

| Analyte | OR (95% CI) |
|---|---|
| CXCL6 | 4.3 (1.9 − 11.7) |
| CASP-8 | 16.1 (3.9 − 107.5) |
| CD40 | 10.1 (3.1 − 49.2) |
| CXCL5 | 2.9 (1.7− 5.7) |
| CXCL1 | 3.9 (1.9 − 9.8) |
| ST1A1 | 6.4 (2.7 − 20.4) |
| EN-RAGE | 5.6 (2.2 − 19.8) |
| Flt3L | 0.036 (0.004 − 0.17) |

Table 6 illustrates that the seven protein analytes with proportionally higher expression in patients with IA were also positively correlated with presence of IA, while the one protein analyte with proportionally lower expression in patients with IA was also negatively correlated with presence of IA, highlighting the predictive power of these protein analytes in detecting and/or classifying IAs in test subjects.

Altogether, the disclosed methods and examples indicate that an immunoassay (e.g., Olink Proximity Extension Assay) can be performed on liquid biological samples (e.g., blood plasma samples) to identify a substantial number of individual plasma proteins nominally associated with the presence of unruptured intracranial aneurysms. A distinct group of analytes were shown to be highly related to presence of unruptured intracranial aneurysms, with medium-to-large effect sizes. Rather than utilize these markers alone, univariate regression, multivariate regression, and Support Vector Machine algorithms were used to identify a multi-protein signature that could reliably distinguish presence of aneurysm and predict presence of intracranial aneurysm on a testing cohort.

### Example 2: Biomarkers for Prediction of Intracranial Aneurysms

CXCL6

Patients diagnosed with unruptured IAs were 4.3 times more likely to exhibit chemokine ligand 6 (CXCL6) in peripheral blood (OR 4.3, 95% CI 1.9—11.7). CXCL6, also known as Granulocyte Chemotactic Protein-2 (GCP-2), is a chemoattractant for neutrophils which plays a role in inflammation and the immune response. As an ELR-containing CXC chemokine, CXCL6 has also been shown to promote angiogenesis and vascular remodeling. Encouragingly, these results are in line with previous evidence that emphasizes the importance of inflammation in IA formation. Specifically, Shi et al. showed an increase in CXCL6 gene expression in IA wall tissue compared to normal superficial temporal artery (STA) tissue (p=0.045036). Using a rabbit IA model, Holcomb et al. showed downregulation of miR-1 which is predicted to target CXCL6 (p=0.0000462). Finally, CXCL6 may be induced by turbulent flow with wall shear stress on IA endothelial cells. See, Proost et al., J Immunol 150, 1000-1010 (1993); Strieter et al., J Biol Chem 270, 27348-27357 (1995); Keeley et al., Arterioscler Thromb Vasc Biol 28, 1928-1936 (2008); Kanematsu et al., Stroke 42, 173-178 (2011); Holcomb et al., AMR Am J Neuroradiol 36, 1710-1715 (2015); and Aoki et al., Acta Neuropathol Commun 4, 48 (2016).

CASP8

Caspase-8 was also highly associated with the presence of unruptured intracranial aneurysms (OR 16.1, 95% CI 3.9—107.5). Caspase-8 is a cysteine protease that initiates extrinsic apoptosis in response to cell surface receptors. The protease is activated by inflammatory cell-derived cytokines and ligands such as tumor necrosis factor alpha and Fas ligand. Caspase-8 has also been shown to modulate cell adhesion and migration. Caspase-8 expression was shown to increase with injury in both rat and dog SAH models. A 2019 study demonstrated that prevention of abdominal aortic aneurysms was mediated in part by down-regulation of caspase-8. See, Muzio et al., Cell 85, 817-827 (1996); Huerta et al., J Surg Res 139, 143-156 (2007); Graf et al., Curr Mot Med 14, 246-254 (2014); Cahill et al., Stroke 37, 1868-1874 (2006); Zhou et al., J Cereb Blood Flow Metab 24, 419-431 (2004); and Liu et al., Cardiovasc Res 115, 807-818 (2019).

CD40

Another correlate, CD40 (OR 10.1, 95% CI 3.1-49.2), is a co-stimulatory membrane protein found on antigen presenting cells and endothelial cells. In dendritic cells, CD40 ligation induces more effective antigen presentation, T-cell stimulatory capacity, and production of several inflammatory cytokines and chemokines. Clinically, CD40 has been shown to play a critical role in autoimmune diseases such as rheumatoid arthritis. It has been indicated that blocking CD40L limits atherosclerosis in mice. Chen et al. identified a correlation between CD40/CD40L mRNA and protein expression levels in humans and coronary heart disease. Increased circulating CD40 ligand levels have been reported to be associated with severity and mortality of severe traumatic brain injury. Importantly, plasma CD40 levels are upregulated in ischemic stroke. Deficiency CD40 ligand was described to protect against aneurysm formation. Studies on aneurysmal subarachnoid hemorrhage have found that increased levels of CD40 and proposed CD40 to be a potential prognostic biomarker of aSAH. See, Schonbeck and Libby, Cell Mol Life Sci 58, 4-43 (2001); Pinchuk et al., Immunity 1, 317-325 (1994); Cella et al., J Exp Med 184, 747-752 (1996); Criswell, Immunol Rev 233, 55-61 (2010); Doran and Veale, Rheumatology (Oxford) 47 Suppl 5, v36-38 (2008); Lutgens et al., Nat Med 5, 1313-1316 (1999); Mach et al., Nature 394, 200-203 (1998); Chen et al., Medicine (Baltimore) 96, e7634 (2017); Lorente et al., Thromb Res 134, 832-836 (2014); Garlichs et al., Stroke 34, 1412-1418 (2003); Ferro et al., Arterioscler Thromb Vasc Biol 27, 2763-2768 (2007); Davi et al., J Atheroscler Thromb 16, 707-713 (2009); Kusters et al., Arterioscler Thromb Vasc Biol 38, 1076-1085 (2018); and Chen et al., Thromb Res 136, 24-29 (2015).

CXCL5

CXCL5 (OR 2.9, 95% CI 1.7—5.7) is produced by immune and vascular endothelial cells in response to proinflammatory cytokines. Like CXCL6, CXCL5 (also known as ENA78) has an ELR motif and is an important chemokine promoter of vascular remodeling. According to a 2016 study, CXCL5 plays a central role as a converging point for upstream infection and downstream neuroinflammation and BBB damage in the pathogenesis of white matter damage in the immature brain. A 2015 study utilizing the Gene Expression Omnibus database identified CXCL5 as a potential precipitator in the pathogenesis of ruptured and unruptured intracranial aneurysm. In the cardiovascular field, CXCL5 is differentially expressed in human aortic aneurysms and has been indicated as a hypertension- and CVD-susceptibility gene. See, Sepuru et al., PLoS One 9, e93228 (2014); Chandrasekar et al., J Biol Chem 278, 4675-4686 (2003); Wang et al., J Neuroinflammation 13, 6 (2016); Zheng et al., Cancer Gene Ther 22, 238-245 (2015); Golledge, Arterioscler Thromb Vasc Biol 33, 670-672 (2013); and Beitelshees et al., Hum Genomics 6, 9 (2012).

CXCL1

CXCL1 (OR 3.9, 95% CI 1.9-9.8) signals via CXCR2 on neutrophils and binds to glycosaminoglycans on endothelial and epithelial cells and the extracellular matrix.

Also known as MGSA and Gro-α in humans and KC in mice, CXCL1 has the ELR motif which associates it with vascular remodeling. Clinical studies and animal models have shown that the chemokine CXCL1 plays dual roles in the host immune response by recruiting and activating neutrophils to combat infection. It directs peripheral neutrophils to the site of infection and then activates the release of proteases and reactive oxygen species (ROS) for microbial killing in the tissue. A 2019 study of aneurysm healing in murine models found CXCL1 decreased murine aneurysm healing after coil implantation. Furthermore, the study showed that therapeutic intervention with a CXCL1 neutralizing antibody enhanced aneurysm healing by decreasing neutrophil infiltration. Recently, Zhao et. al exhibited that both cyclic mechanical stress and abdominal aortic constriction induce CXCL1 expression. See, Sawant et al., Sci Rep 6, 33123 (2016); Cummings et al., J Immunol 162, 2341-2346 (1999); Ritzman et al., Infect Immun 78, 4593-4600 (2010); Jin et al., J Immunol 193, 3549-3558 (2014); Patel et al., Neurosurgery 66, (2019); and Zhao et al., Sci Rep 7, 16128 (2017).

ST1A1/SULT1A1

Sulfotransferase 1A1 (OR 6.4, 95% CI 2.7—20.4) is an established binding site of non-steroidal anti-inflammatory drugs with phenolic structures, such as acetaminophen. One mechanism of sulfonation is defense against certain chemicals via inflammation and elimination from the body. Sulfotransferase (SULT)1A1 is the isoform responsible for the metabolism and subsequent disposition of a number of exogenous substances possessing a small phenolic structure, ST1A1 or SULT1A1. One study on microarrays found that Sult1a1 transcript expression increased two-fold in active multiple sclerosis lesions. Another investigation of micro-dissected white matter astrocytes identified higher sulfotransferase 1A1 expression during autoimmune neuroinflammation. See, Wang et al., J Biol Chem 292, 20305-20312 (2017); Reiter and Weinshilboum, Clin Pharmacol Ther 32, 612-621 (1982); Lock et al., Nat Med 8, 500-508 (2002); and Guillot et al., J Neuroinflammation 12, 130 (2015).

EN-RAGE

EN-RAGE was also strongly predictive in patients with IAs compared with controls (OR 5.6, 95% CI 2.2—19.8). Also known as S100A12, EN-RAGE is a ligand that binds to RAGE and activates pro-inflammatory genes. The EN-RAGE inflammatory pathway has been linked to a wide range of diseases, such as atherosclerosis, rheumatoid arthritis, and Alzheimer's disease. A study on aortic aneurysms in transgenic mice concluded that EN-RAGE is sufficient to activate pathogenic pathways through the modulation of oxidative stress, inflammation and vascular remodeling in vivo, leading to aortic wall remodeling and aortic aneurysm. Furthermore, a 2014 study found that higher EN-RAGE levels were significantly correlated with an increased risk of congenital heart disease beyond conventional risk factors. See, Hofmann et al., Cell 97, 889-901 (1999); Schmidt et al., J Clin Invest 108, 949-955 (2001); Foell et al., Rheumatology (Oxford) 42, 1383-1389 (2003); Emanuele et al., Arch Neurol 62, 1734-1736 (2005); Hofmann Bowman et al., Circ Res 106, 145-154 (2010); and Ligthart et al., Arterioscler Thromb Vasc Biol 34, 2695-2699 (2014).

FIt3L/Flt1

Interestingly, FIt3L was inversely correlated with UIAs (OR=0.036, CI 0.004—0.17). FIt3L (Fms-related tyrosine kinase 3 ligand) is a hematopoietic factor that can be used as an immunomodulatory agent. FIt3L specifically expands early hematopoietic stem cells by acting on the class III tyrosine kinase receptor, Flt3R, which is expressed predominantly on hematopoietic progenitor cells. Flt3L is typically a cell surface transmembrane protein that can also be proteolytically cleaved and released as a soluble protein.

It has been described as a growth factor and a key regulator of dendritic cell homeostasis. In opposition with the seven previously identified proteins that comprise our IA proteomic signature, Flt3L has been shown to decrease the levels of programmed cell death in dendritic cells and macrophages. Therefore, it is plausible that FIt3L may exhibit protective effects from aneurysm formation. See, Lyman, Int Hematol 62, 63-73 (1995); Gabbianelli et al., Blood 86, 1661-1670 (1995); Liu and Nussenzweig, Immunol Rev 234, 45-54 (2010); and Patil et al., Shock 47, 40-51 (2017).

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for detecting an intracranial aneurysm in a test subject, comprising:
obtaining one or more liquid biological samples from the test subject, wherein each liquid biological sample in the one or more liquid biological samples comprises a plurality of protein analytes;
analyzing each liquid biological sample in the one or more liquid biological samples using an immunoassay, thereby obtaining a test dataset comprising a plurality of abundance measures, wherein each abundance measure in the plurality of abundance measures corresponds to a respective protein analyte in the plurality of protein analytes in each respective liquid biological sample in the one or more liquid biological samples; and
inputting the test dataset into a trained classifier, thereby obtaining an indication from the trained classifier that the subject has an intracranial aneurysm, based at least in part on the plurality of abundance measures for the test subject in the test dataset.

2. The method of claim 1, wherein the analyzing each liquid biological sample using an immunoassay comprises measuring the abundance of one or more protein analytes selected from a predefined panel of protein analytes.

3. The method of claim 2, wherein the predefined panel of protein analytes comprises one or more analytes selected from Table 1.

4. The method of claim 2, wherein the predefined panel of protein analytes comprises one or more analytes selected from Table 2.

5. The method of any one of claims 1-4, wherein the immunoassay is a high-throughput multiplex proximity extension immunoassay.

6. The method of any one of claims 1-5, wherein:
the test dataset further comprises a first label indicating a corresponding first covariate for the test subject,
the indication from the trained classifier that the subject has an intracranial aneurysm is further based on the first covariate, and
the corresponding first covariate is selected from the group consisting of:
an age of the test subject;
a sex of the test subject;
a hypertension status;
a hyperlipidemia status;
a presence or absence of diabetes mellitus type II;
a smoking history; and
any combination thereof.

7. The method of any one of claims 1-6, wherein the test dataset is pre-processed by normalization of the plurality of abundance measures prior to the inputting the test dataset into the trained classifier.

8. The method of any one of claims 1-7, wherein the test dataset is processed, prior to the inputting the test dataset into the trained classifier, by removing from the dataset one or more protein analytes that fail to meet one or more selection criteria.

9. The method of claim 8, wherein the one or more selection criteria is a threshold limit of detection.

10. The method of claim 8, wherein the one or more selection criteria is inclusion in a predefined panel of protein analytes.

11. The method of claim 1, wherein the indication comprises a probability that the subject has an intracranial aneurysm and a prediction of a size of an intracranial aneurysm.

12. The method of any one of claims 1-11, wherein the trained classifier is a neural network algorithm, a support vector machine algorithm, a Naïve Bayes algorithm, a decision tree algorithm, an unsupervised clustering model algorithm, a supervised clustering model algorithm, or a regression model.

13. The method of any one of claims 1-12, wherein the test subject is a human.

14. The method of any one of claims 1-13, wherein the test subject has an unruptured intracranial aneurysm.

15. The method of any one of claims 1-14, wherein each liquid biological sample in the one or more liquid biological samples is a blood sample.

16. The method of any one of claims 1-15, wherein each abundance measure in the plurality of abundance measures is a relative protein concentration.

17. The method of any one of claims 1-16, wherein the obtaining one or more liquid biological samples from the test subject is performed by venipuncture.

18. The method of any one of claims 1-17, the method further comprising:
applying a treatment regimen to the test subject based at least in part, on the indication.

19. The method of claim 18, wherein the treatment regimen comprises applying an agent for intracranial aneurysm.

20. The method of claim 19, wherein the agent for intracranial aneurysm is a hormone, an immune therapy, radiography, or a drug.

21. The method of any one of claims 1-17, wherein the subject has been treated with an agent for intercranial aneurysm and the method further comprises:
using the indication to evaluate a response of the test subject to the agent for intercranial aneurysm.

22. The method of claim 21, wherein the agent for intercranial aneurysm is a hormone, an immune therapy, radiography, or a drug.

23. The method of any one of claims 1-17, wherein the subject has been treated with an agent for intercranial aneurysm and the method further comprises:
using the indication to determine whether to intensify or discontinue the agent for intercranial aneurysm in the test subject.

24. The method of any one of claims 1-17, wherein the subject has been subjected to a surgical intervention to address the intercranial aneurysm and the method further comprises:
using the indication to assess a success of the surgical intervention.

25. A device for detecting an intracranial aneurysm in a test subject, comprising one or more processors, and memory storing one or more programs for execution by the one or more processors, the one or more programs comprising instructions for:
obtaining one or more liquid biological samples from the test subject, wherein each liquid biological sample in the one or more liquid biological samples comprises a plurality of protein analytes;
analyzing each liquid biological sample in the one or more liquid biological samples using an immunoassay, thereby obtaining a test dataset comprising a plurality of abundance measures, wherein each abundance measure in the plurality of abundance measures corresponds to a respective protein analyte in the plurality of protein analytes in each respective liquid biological sample of the test subject in the one or more liquid biological samples; and
inputting the test dataset into a trained classifier, thereby obtaining an indication from the trained classifier that the subject has an intracranial aneurysm, based at least in part on the plurality of abundance measures for the test subject in the test dataset.

26. A non-transitory computer readable storage medium and one or more computer programs embedded therein for classification, the one or more computer programs comprising instructions which, when executed by a computer system, cause the computer system to perform a method for detecting an intracranial aneurysm in a test subject, the method comprising:
obtaining one or more liquid biological samples from the test subject, wherein each liquid biological sample in the one or more liquid biological samples comprises a plurality of protein analytes;
analyzing each liquid biological sample in the one or more liquid biological samples using an immunoassay, thereby obtaining a test dataset comprising a plurality of abundance measures, wherein each abundance measure in the plurality of abundance measures corresponds to a respective protein analyte in the plurality of protein analytes in each respective liquid biological sample in the one or more liquid biological samples; and
inputting the test dataset into a trained classifier, thereby obtaining an indication from the trained classifier that the subject has an intracranial aneurysm, based at least in part on the plurality of abundance measures for the test subject in the test dataset.

27. A classification method comprising,
at a computer system having one or more processors, and memory storing one or more programs for execution by the one or more processors:
for each training subject in a plurality of training subjects, wherein each training subject in the plurality of training subjects is distinguished as having a first diagnostic status corresponding to either a presence of an intracranial aneurysm or an absence of an intracranial aneurysm, obtaining one or more liquid biological samples from each respective training subject, thereby obtaining a plurality of liquid biological samples, wherein each liquid biological sample comprises a plurality of protein analytes;
analyzing each liquid biological sample in the plurality of liquid biological samples using an immunoassay, thereby obtaining a first dataset comprising, for each training subject in the plurality of training subjects:
(i) a first label indicating the corresponding first diagnostic status of the respective subject; and
(ii) a plurality of abundance measures, wherein each abundance measure in the plurality of abundance measures corresponds to a respective protein analyte in the plurality of protein analytes in each respective liquid biological sample in the one or more liquid biological samples; and
training an untrained or partially untrained classifier with the first dataset, thereby obtaining a trained classifier that provides an indication that a subject has an intracranial aneurysm, based at least in part on a plurality of abundance measures for a corresponding plurality of protein analytes in one or more liquid biological samples of the subject.

28. The method of claim 27, wherein the analyzing each liquid biological sample using an immunoassay comprises measuring the abundance of one or more protein analytes selected from a predefined panel of protein analytes.

29. The method of claim 28, wherein the predefined panel of protein analytes comprises one or more analytes selected from Table 1.

30. The method of claim 28, wherein the predefined panel of protein analytes comprises one or more analytes selected from Table 2.

31. The method of any one of claims 27-30, wherein the immunoassay is a high-throughput multiplex proximity extension immunoassay.

32. The method of any one of claims 27-31, wherein:
the plurality of training subjects comprises a first subset of training subjects and a second subset of training subjects;
each respective training subject in the first subset of training subjects has a first diagnostic status corresponding to a presence of an intracranial aneurysm;
each respective training subject in the second subset of training subjects has a first diagnostic status corresponding to an absence of an intracranial aneurysm; and
the number of training subjects in the first subset of training subjects is equal to the number of training subjects in the second subset of training subjects.

33. The method of any one of claims 27-32, wherein the first dataset is pre-processed by normalization of the plurality of abundance measures prior to the training the untrained or partially untrained classifier with the first dataset.

34. The method of any one of claims 27-33, wherein the first dataset is processed, prior to the training the untrained or partially untrained classifier with the first dataset, by removing from the dataset one or more protein analytes that fail to meet one or more selection criteria.

35. The method of claim 34, wherein the one or more selection criteria is a threshold limit of detection.

36. The method of claim 34, wherein the one or more selection criteria is inclusion in a predefined panel of protein analytes.

37. The method of claim 34, wherein the one or more selection criteria is a threshold p-value, wherein the p-value for each one or more protein analyte is (i) determined using a significance test and (ii) calculated over the plurality of abundance measures corresponding to the respective protein analyte across the plurality of training subjects.

38. The method of claim 37, wherein the significance test is a univariate linear regression model, a univariate logistic regression model, a multivariate linear regression model, a multivariate logistic regression model, a chi-squared test, Fishers Exact test, Student's t-test, or a binary proportional test.

39. The method of claim 37, wherein the threshold p-value is 0.05.

40. The method of claim 37, wherein the threshold p-value is 0.0001.

41. The method of any one of claims 27-40, wherein the first dataset further comprises, for each subject in the plurality of subjects, a second label indicating a corresponding second diagnostic status, wherein the second diagnostic status is selected from the group consisting of:
a size of an intracranial aneurysm;
a location of an intracranial aneurysm;
a presence or absence of aneurysmal rupture;
a saccular aneurysm;
an endovascular treatment status for an intracranial aneurysm;
an open treatment status for an intracranial aneurysm;
an age of a training subject;
a sex of a training subject;
a hypertension status;
a hyperlipidemia status;
a presence or absence of diabetes mellitus type II;
a smoking history; and
any combination thereof.

42. The method of claim 41, wherein the indication from the trained classifier that a subject has an intracranial aneurysm is further based on the second diagnostic status.

43. The method of claim 41, wherein the trained classifier further provides an indication that a subject has the second diagnostic status.

44. The method of claim 43, wherein the indication comprises a probability that a subject has an intracranial aneurysm and a prediction of a size of an intracranial aneurysm.

45. The method of any one of claims 27-44, wherein the trained classifier is a neural network algorithm, a support vector machine algorithm, a Naïve Bayes algorithm, a decision tree algorithm, an unsupervised clustering model algorithm, a supervised clustering model algorithm, or a regression model.

46. The method of any one of claims 27-45, wherein, prior to the training the untrained or partially untrained classifier, the performance of the untrained or partially untrained classifier is validated on the first dataset using k-fold cross validation.

47. The method of claim 46, wherein k is between 2 and 60.

48. The method of any one of claims 27-47, wherein each training subject in the plurality of training subjects is a human.

49. The method of any one of claims 27-48, wherein each liquid biological sample in the plurality of liquid biological samples is a blood sample.

50. The method of any one of claims 27-49, wherein each abundance measure in the plurality of abundance measures is a relative protein concentration.

51. The method of any one of claims 27-50, wherein the obtaining one or more liquid biological samples from each respective training subject is performed by venipuncture.

52. A classification device comprising one or more processors, and memory storing one or more programs for execution by the one or more processors, the one or more programs comprising instructions to perform a classification method comprising:
for each training subject in a plurality of training subjects, wherein each training subject in the plurality of training subjects is distinguished as having a first diagnostic status corresponding to either a presence of an intracranial aneurysm or an absence of an intracranial aneurysm, obtaining one or more liquid biological samples from each respective training subject, thereby obtaining a plurality of liquid biological samples, wherein each liquid biological sample comprises a plurality of protein analytes;
analyzing each liquid biological sample in the plurality of liquid biological samples using an immunoassay, thereby obtaining a first dataset comprising, for each training subject in the plurality of training subjects:
(i) a first label indicating the corresponding first diagnostic status of the respective subject; and
(ii) a plurality of abundance measures, wherein each abundance measure in the plurality of abundance measures corresponds to a respective protein analyte in the plurality of protein analytes in each respective liquid biological sample in the one or more liquid biological samples; and
training an untrained or partially untrained classifier with the first dataset, thereby obtaining a trained classifier that provides an indication that a subject has an intracranial aneurysm, based at least in part on a plurality of abundance measures for a respective plurality of protein analytes in one or more liquid biological samples of the subject.

53. A non-transitory computer readable storage medium and one or more computer programs embedded therein for classification, the one or more computer programs comprising instructions which, when executed by a computer system, cause the computer system to perform a classification method comprising:
for each training subject in a plurality of training subjects, wherein each training subject in the plurality of training subjects is distinguished as having a first diagnostic status corresponding to either a presence of an intracranial aneurysm or an absence of an intracranial aneurysm, obtaining one or more liquid biological samples from each respective training subject, thereby obtaining a plurality of liquid biological samples, wherein each liquid biological sample comprises a plurality of protein analytes;
analyzing each liquid biological sample in the plurality of liquid biological samples using an immunoassay, thereby obtaining a first dataset comprising, for each training subject in the plurality of training subjects:
(i) a first label indicating the corresponding first diagnostic status of the respective subject; and
(ii) a plurality of abundance measures, wherein each abundance measure in the plurality of abundance measures corresponds to a respective protein analyte in the plurality of protein analytes in each respective liquid biological sample in the one or more liquid biological samples; and
training an untrained or partially untrained classifier with the first dataset, thereby obtaining a trained classifier that provides an indication that a subject has an intracranial aneurysm, based at least in part on a plurality of abundance measures for a respective plurality of protein analytes in one or more liquid biological samples of the subject.
